(19) United States
(12) Patent Application Publication
(10) Pub. No.: US 2025/0312173 A1
(43) Pub. Date: Oct. 9, 2025

(54) ADAPTABLE PROSTHESIS

(71) Applicant: GroLimb, LLC, Iowa City, IA (US)

(72) Inventors: Cláudia Nicole Archer, Omaha, NE (US); Claire Elizabeth Catlett, Johnston, IA (US); Madelyn Rose Laura Timm, Stewartville, MN (US); Tushar Arjun Mitra, Downers Grove, IL (US)

(21) Appl. No.: 19/173,963

(22) Filed: Apr. 9, 2025

Related U.S. Application Data
(60) Provisional application No. 63/631,769, filed on Apr. 9, 2024.

(51) Int. Cl. A61F 2/80; A61F 2/60; A61F 2/78

(52) U.S. Cl. CPC A61F 2/80; A61F 2002/607; A61F 2002/7887

(57) ABSTRACT

A prosthesis is provided. The proximal end of an adjustable socket engages the residual limb of a user. The girth of the socket can be adjusted using a flexible locking mechanism such as a dial, overlapping flaps, or a ratchet system. The distal end of the socket attaches to the proximal end of an adjustable pylon comprising a rack and lockrack or rack and pinion structure. The rack inserts into a guiderail, and linear movement of the rack allows the pylon to be lengthened as the user grows. The rack is held in position to the guiderail using a lockrack. The distal end of the pylon attaches to an adjustable foot comprising a base that mimics the shape of a human foot. The size of the foot can be adjusted using at least one expansion member as the user grows.

### CROSS-REFERENCE TO RELATED APPLICATIONS

This patent application claims priority to U.S. Provisional Patent Application No. 63/631,769 filed on Apr. 9, 2024, the entirety of which is incorporated herein fully by reference.

### FIELD OF THE DISCLOSURE

This disclosure relates generally to a prosthetic device, and in particular to an adaptable below-the-knee prosthesis.

### BACKGROUND OF THE DISCLOSURE

A prosthesis is a device designed to replace a missing part of the body or to make a part of the body work better. Currently, as a child or user who uses a prosthesis grows, the prosthesis will need to be periodically replaced to match the growth of the child or user. Thus, prosthetic devices for children or other users can be very expensive, create an abundance of material waste, and require many appointments.

Attempts to create an adjustable below-the-knee prosthetic have been made. One such attempt, developed by Martin Bionics Prosthetics, is the “Modular Socket-less Socket”. This socket, which is a part of the prosthetic device, allows for adjustability without requiring intricate fabrication equipment or the expertise of a prosthetist. However, a notable limitation of this design is its minimal contact points, potentially leading to increased stress and pressure on the residual limb. Further, the design of an “Infinite Socket” involves four struts that can be adjusted with a heat gun and an adjustable lanyard system. This design may improve a prosthetic's shock absorption and pressure distribution, but cannot adjust to be compatible with the limb lengths of pediatric patients. As such, while the “Infinite Socket” and “Modular Socket-less Socket” are effective for adult populations, they may not adequately address the height or width adjustment requirements essential for the pediatric demographic.

Another adjustable prosthetic is seen in the “Ossur Connect TF Prosthetic Socket” which offers an adjustable socket that can be fitted in under a day. While this design allows some adjustability, it requires a large amount of time for fitting and is designed specifically for the adult population. Therefore, it is not effective for pediatric patients.

Additionally, the Click Medical RevoFit design allows for adjustments in the girth of the socket of the prosthetic, using a Click Reel. Further, Hanger Clinic's ComfortFlex Adapt, the Adjustable Prosthesis filed by Empowering Engineering Technologies Corporation (WO2010129334A2), and the iFit Prosthetics' Transtibial System all have the same adjustability purpose as the Click Medical RevoFit, but with alternate mechanisms and methods. In their own forms, these devices only adapt in width and do not allow for vertical extension of the prosthetic. Vertical adjustability is not considered in these devices because these socket examples are intended for adult amputees with volume fluctuation, but not necessarily developmental growth.

Another available and noteworthy device is the Nonspec product, designed for adjustments in the pylon of a prosthetic. This device allows for height and gait adjustments with minimal training required for users; however, the limb diameter and size of the foot cannot be altered.

Additionally, another recognized device is the Össur Height-Adjustable Pylon, which enables convenient adjustments to height and rotation of a prosthetic limb. This pylon is available in three different sizes, including two adult versions and one junior version. Of particular note, the junior version is only designed to support users in a particular weight and height range. As such, this device is limited to users in those ranges. Further, the device adjusts to the user's varying sizes using rotational increments which adds complexity to the adjustment process.

As such, the prior attempts described above focus on adjusting different segments of a prosthesis and/or use complex adjustment processes, but none allow for the easy modification of both the height and width of the prosthetic. Specifically, many older attempts require the replacement of whole pieces rather than the adjustment of them, such as the Prosthetic Leg filed by Flex Foot (U.S. Pat. No. 5,725,598A). The length adjustment in this design focuses on flexibility of the pylon for the sake of gait and energy return. This is not ideal for pediatric applications as it does not allow for fine and convenient adjustment in length. Additionally, the connection of this pylon to the socket and foot pieces is not standard for current component adapter pieces.

Also, most prior attempts for prosthetic feet do not allow for adjustments in length or width, such as the Prosthetic Foot filed by Ossur (U.S. Pat. No. 8,177,855B2). However, there are a couple of prior attempts that offer some adjustment capabilities. One example of this is the Adjustable Length Prosthetic Foot filed by Otto Bock Healthcare (EP3131505). This device has three components, including a spring element, an attachment member, and a heel member. The points of attachment include slots that allow for some extension in length but no adaptability in width. An additional device to note is the Prosthetic Foot with Fully Adaptable Hindfoot and Forefoot Keels filed by Matthew Habecker (US20060235545A1). The adaptable keels are designed to allow for more control of the inversion/eversion of a foot as well as pronation/supination of a foot. However, while this design may be beneficial for the comfort and stability of patients, it does not address the need for length and width adaptability.

For the reasons stated above, and for other reasons which will become apparent to those skilled in the art upon reading and understanding the specification, there is a need in the art for a way to simplify the adaptive fit process of a prosthesis in response to growth.

These and other objects, features, or advantages of the present disclosure will become apparent from the specification and claims.

### BRIEF SUMMARY OF THE DISCLOSURE

The disclosure relates to an adaptable prosthesis. In one arrangement, the prosthesis comprises a socket configured to engage the residual limb of a child or user. A dial, ratchet system, or other fastening mechanism allows adjustability of the socket. A lower end of the socket securely attaches to an adjustable pylon. The pylon comprises a rack and pinion structure or a concealed rack system where the rack inserts into a guiderail and linear movement of the rack allows the pylon to be lengthened as the child or user grows. The rack is held in position using a lockrack. Further, a lower end of the adjustable pylon attaches to a foot. The foot comprises a base that mimics the shape of a natural human foot and at least one expansion layer to adjust the width and length of the foot.

The prosthesis' components are designed to be adjustable in all relevant directions, while maintaining certain functional requirements. The prosthesis facilitates typical biomechanics, including full range of motion at the knee/hip and allows for proper gait without exertion. The design is also comparable in weight to existing solutions (less than 4 pounds). The prosthesis is easy to use without professional clinical training and is usable without sophisticated tools. In addition, the device is comfortable and durable. In terms of cost efficiency, the device is affordable because it uses materials that are simple to process and allows for the reduction of prosthesis reiteration frequency.

### BRIEF DESCRIPTION OF THE DRAWINGS

FIG. 1 depicts a right side view of a prosthesis according to one embodiment.

FIG. 2 depicts an anterior or front side view of a prosthesis according to one embodiment.

FIG. 3 depicts a left side view of a prosthesis according to one embodiment.

FIG. 4 depicts a posterior or back side view of a prosthesis according to one embodiment.

FIG. 5 depicts a top side view of a prosthesis according to one embodiment.

FIG. 6 depicts a bottom side view of a prosthesis according to one embodiment.

FIG. 7 depicts a right side view of a prosthesis according to one embodiment.

FIG. 8 depicts an anterior or front side view of a prosthesis according to one embodiment.

FIG. 9 depicts a left side view of a prosthesis according to one embodiment.

FIG. 10 depicts a posterior or back side view of a prosthesis according to one embodiment.

FIG. 11 depicts a top side view of a prosthesis according to one embodiment.

FIG. 12 depicts a bottom side view of a prosthesis according to one embodiment.

FIG. 13A depicts a pin of a pin locking liner suspension system of a prosthesis according to one embodiment.

FIG. 13B depicts a lock of a pin locking liner suspension system of a prosthesis according to one embodiment.

FIG. 14 depicts a cross section of a pylon gear teeth interface for a prosthesis according to one embodiment.

FIG. 15 depicts a pylon of a prosthesis in a fully extended position according to one embodiment.

FIG. 16 depicts a pylon of a prosthesis in a fully closed or retracted position according to one embodiment.

FIG. 17 depicts a cross section of a pylon of a prosthesis in a fully closed or retracted position according to one embodiment.

FIG. 18 depicts a pylon of a prosthesis in a fully extended position according to one embodiment.

FIG. 19 depicts a pylon of a prosthesis in a fully closed or retracted position according to one embodiment.

FIG. 20 depicts a posterior or back side view of a pylon of a prosthesis according to one embodiment.

FIG. 21 depicts a left side view of a pylon of a prosthesis according to one embodiment.

FIG. 22 depicts a right side view of a pylon of a prosthesis according to one embodiment.

FIG. 23 depicts a top side view of a pylon of a prosthesis according to one embodiment.

FIG. 24 depicts a bottom side view of a pylon of a prosthesis according to one embodiment.

FIG. 25 depicts an exploded view of a pylon of a prosthesis according to one embodiment.

FIG. 26 depicts a top side view of a fully retracted foot of a prosthesis according to one embodiment.

FIG. 27 depicts a top side view of a fully expanded foot of a prosthesis according to one embodiment.

FIG. 28 depicts a top side view of a foot of a prosthesis according to one embodiment.

FIG. 29 depicts a perspective view of a fully retracted foot of a prosthesis according to one embodiment.

FIG. 30 depicts a perspective view of a fully expanded foot of a prosthesis according to one embodiment.

FIG. 31 depicts an exploded view of a foot of a prosthesis according to one embodiment.

FIG. 32 depicts a top side view of a fully retracted foot of a prosthesis according to one embodiment.

FIG. 33 depicts a right side view of a fully retracted foot of a prosthesis according to one embodiment.

FIG. 34 depicts a bottom side view of a fully retracted foot of a prosthesis according to one embodiment.

FIG. 35 depicts a larger top segment of a socket of a prosthesis according to one embodiment.

FIG. 36 depicts a smaller top segment of a socket of a prosthesis according to one embodiment.

FIG. 37 depicts an anterior or front side view of a socket of a prosthesis according to one embodiment.

FIG. 38 depicts a left side view of a socket of a prosthesis according to one embodiment.

FIG. 39 depicts a right side view of a socket of a prosthesis according to one embodiment.

FIG. 40 depicts a posterior or back side view of a socket of a prosthesis according to one embodiment.

FIG. 41 depicts a top side view of an expanded socket of a prosthesis according to one embodiment.

FIG. 42 depicts a top side view of a compacted socket of a prosthesis according to one embodiment.

FIG. 43 depicts an exploded socket of a prosthesis according to one embodiment.

FIG. 44 depicts a liner and padding of a socket of a prosthesis according to one embodiment.

FIG. 45 depicts an exploded view of a prosthesis according to one embodiment.

FIG. 46 depicts an exploded view of a prosthesis according to one embodiment.

FIG. 47 depicts a back perspective view of a prosthesis according to one embodiment.

FIG. 48 depicts a front perspective view of a prosthesis according to one embodiment.

FIG. 49 depicts a back perspective view of a prosthesis according to one embodiment.

FIG. 50 depicts a front perspective view of a prosthesis according to one embodiment.

FIG. 51 depicts a lock of a pin locking liner suspension system of a prosthesis held by a fastener in a socket of a prosthesis according to one embodiment.

FIG. 52 depicts a lock of a pin locking liner suspension system of a prosthesis according to one embodiment.

### DETAILED DESCRIPTION

In the following detailed description of the embodiments, reference is made to the accompanying drawings which form a part hereof, and in which is shown by way of illustration specific preferred embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the present disclosures. The following detailed description is, therefore, not to be taken in a limiting sense, and the scope of the present disclosure is defined only by the appended claims, along with the full scope of equivalents to which such claims are entitled.

As used herein, the terminology such as vertical, horizontal, top, bottom, front, back, end and sides are referenced according to the views presented. It should be understood, however, that the terms are used only for purposes of description and are not intended to be used as limitations. Accordingly, orientation of an object or a combination of objects may change without departing from the scope of the disclosure.

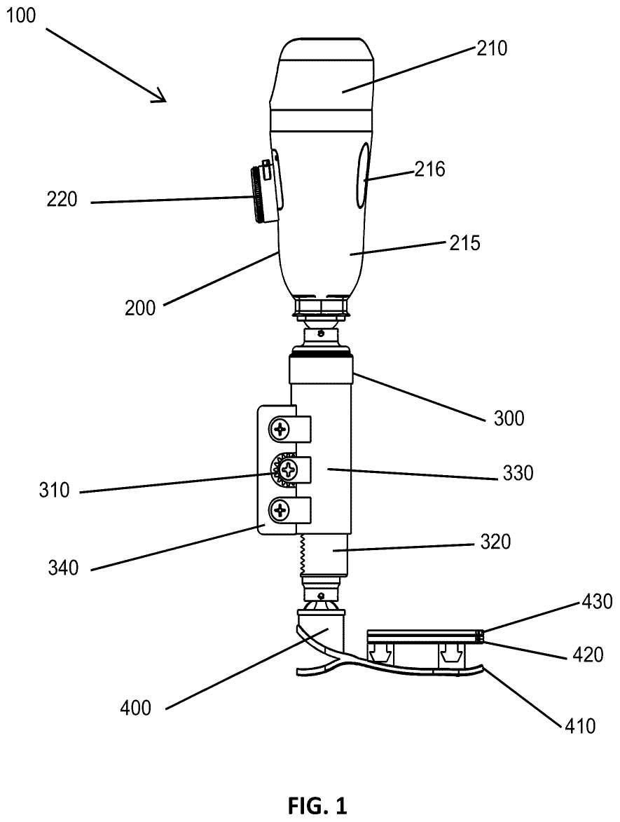

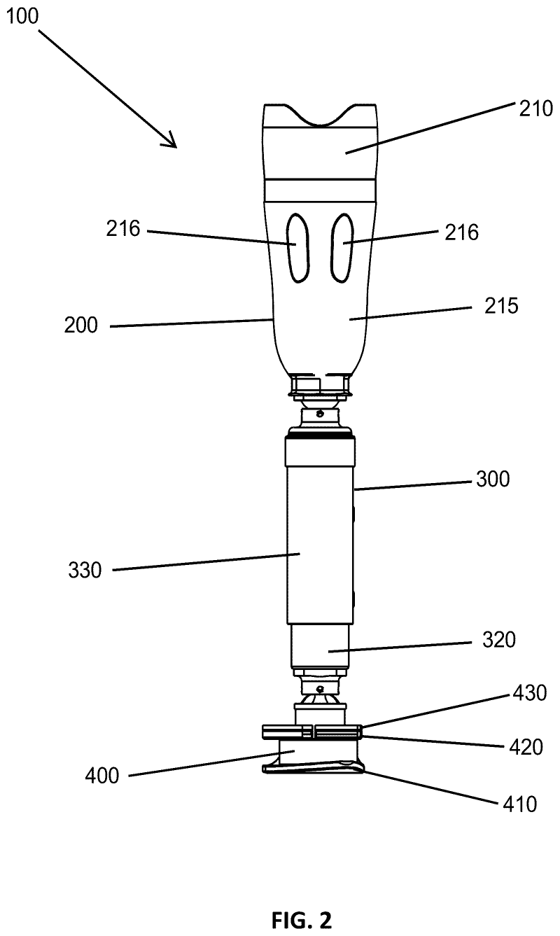

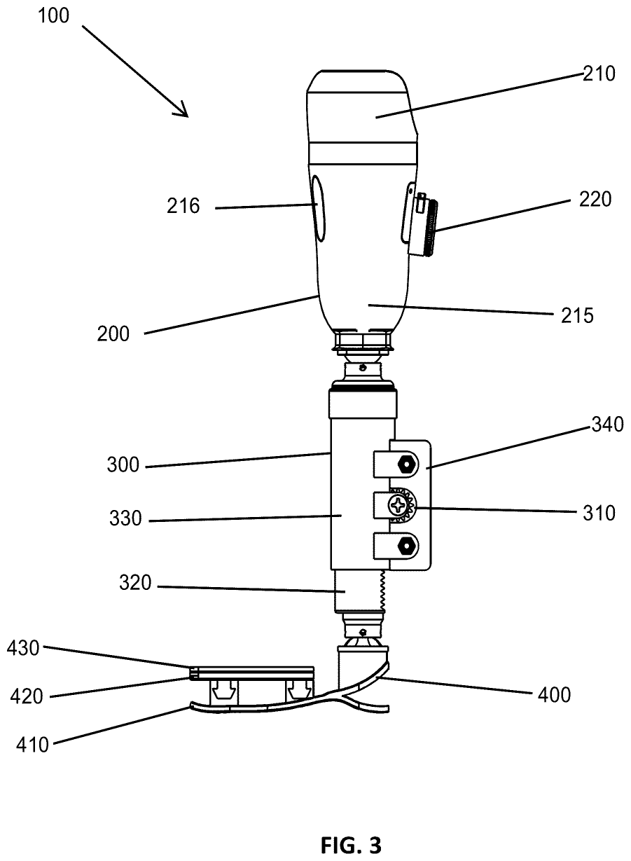

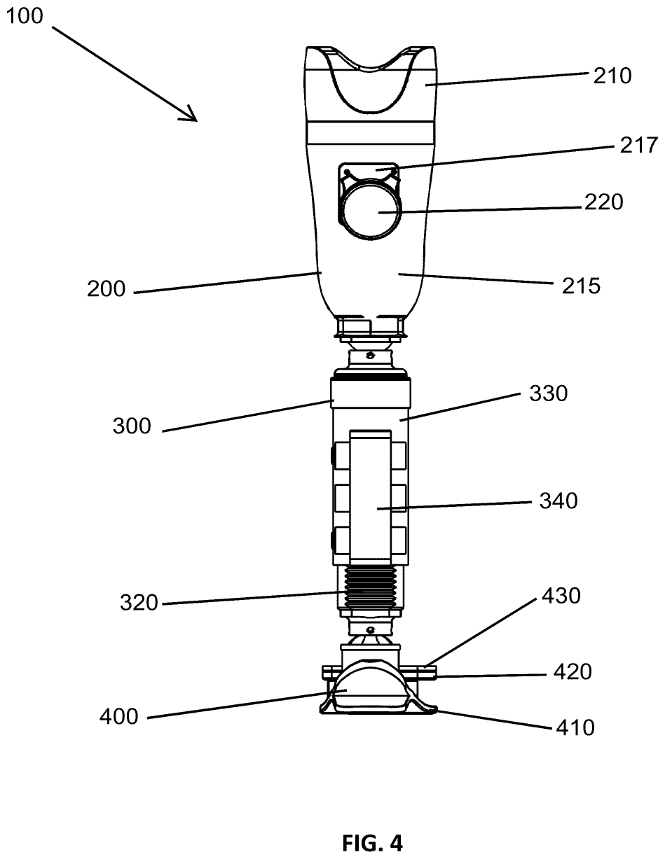

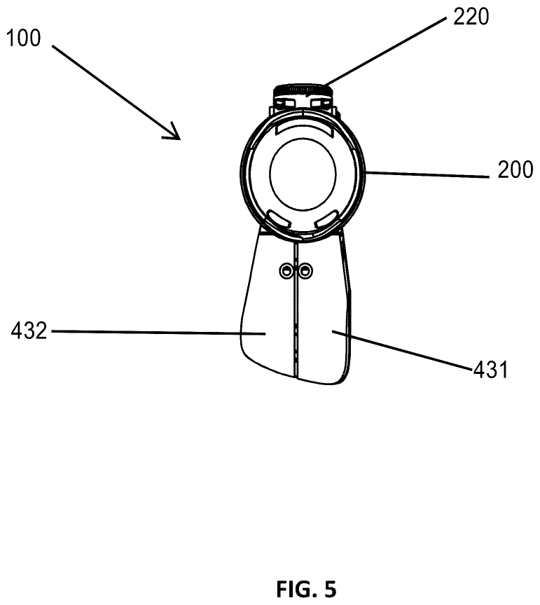

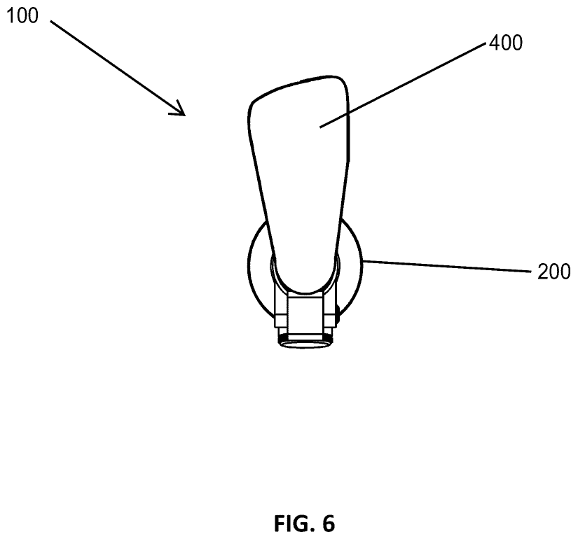

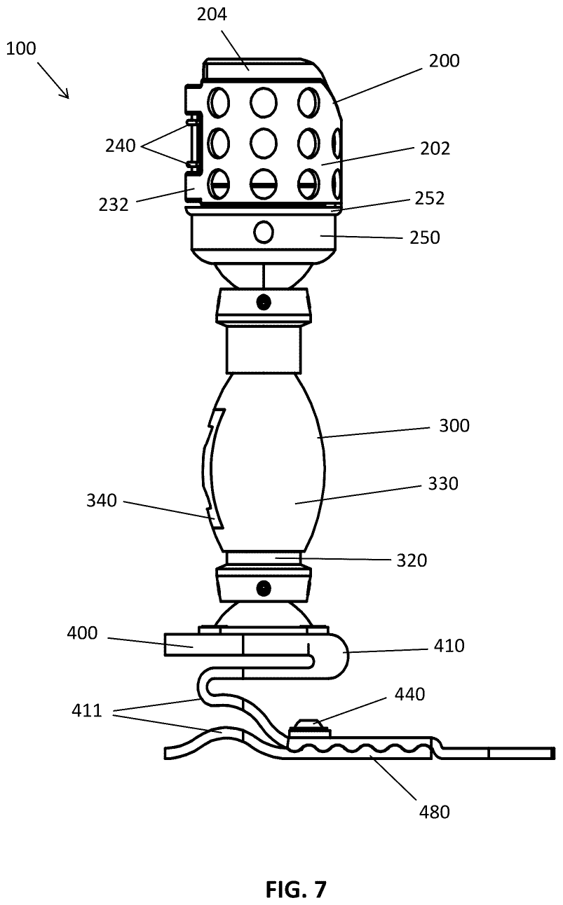

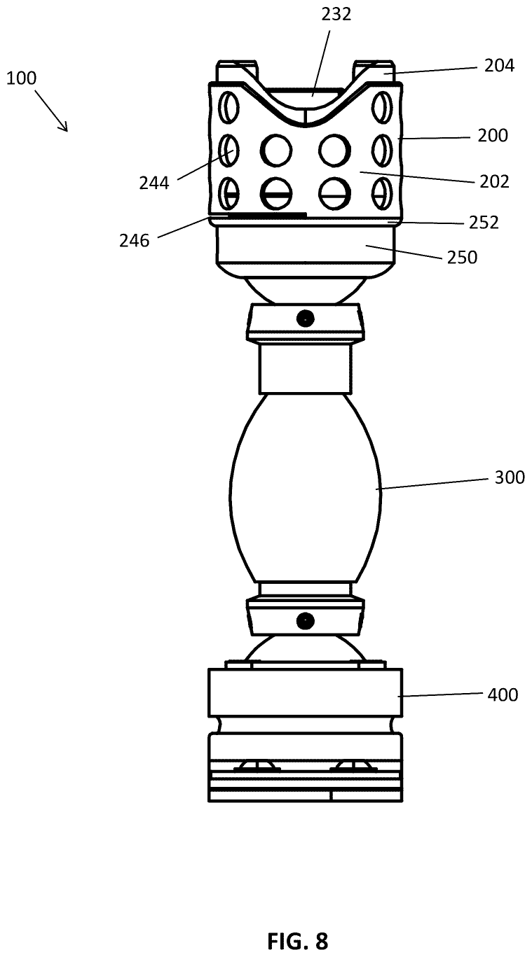

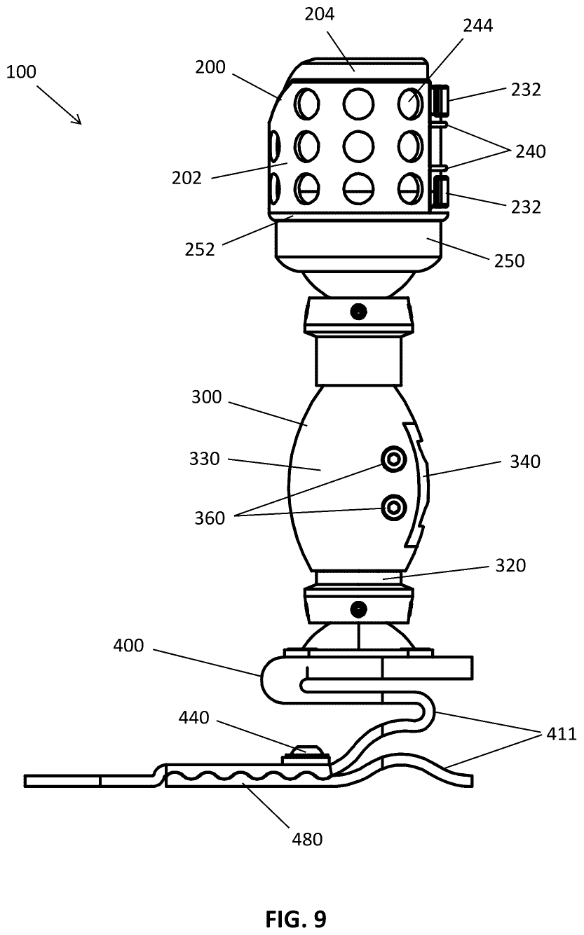

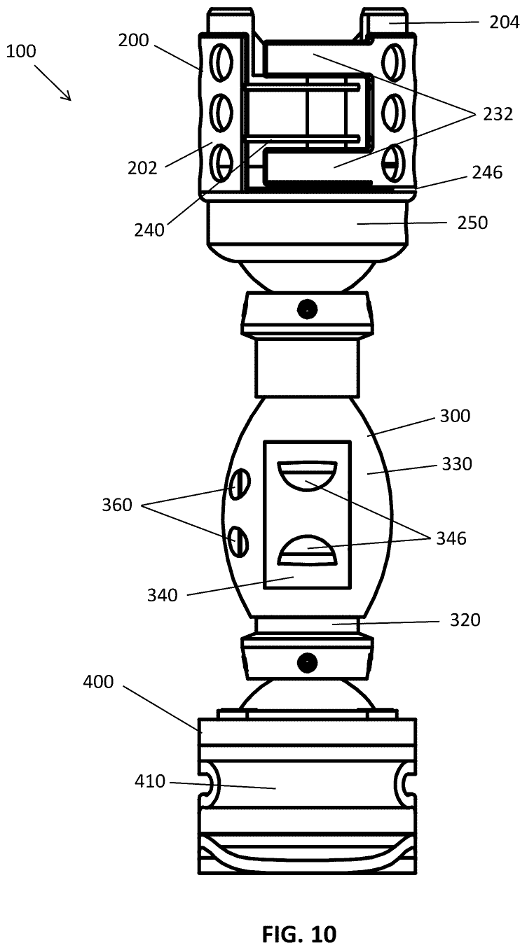

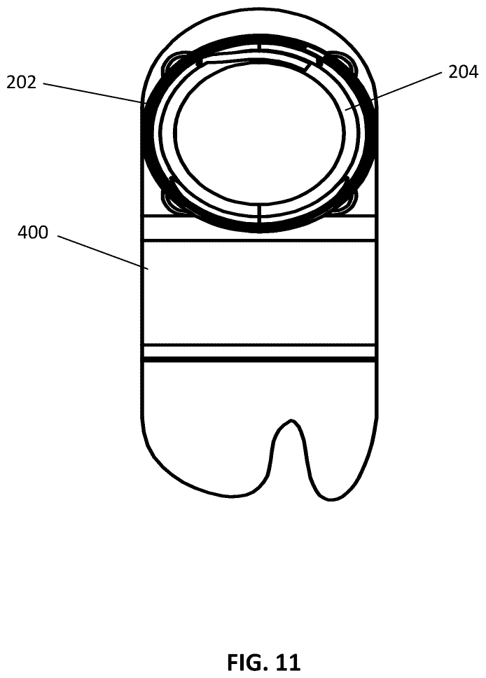

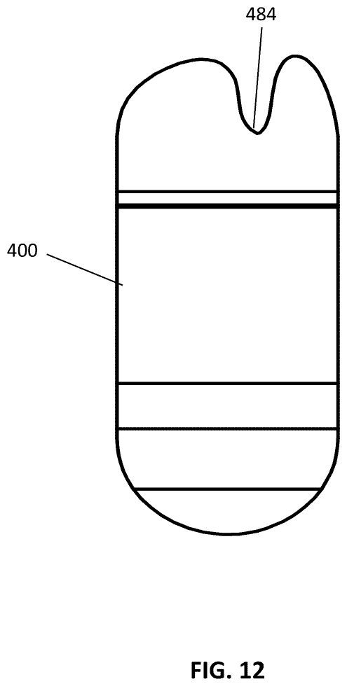

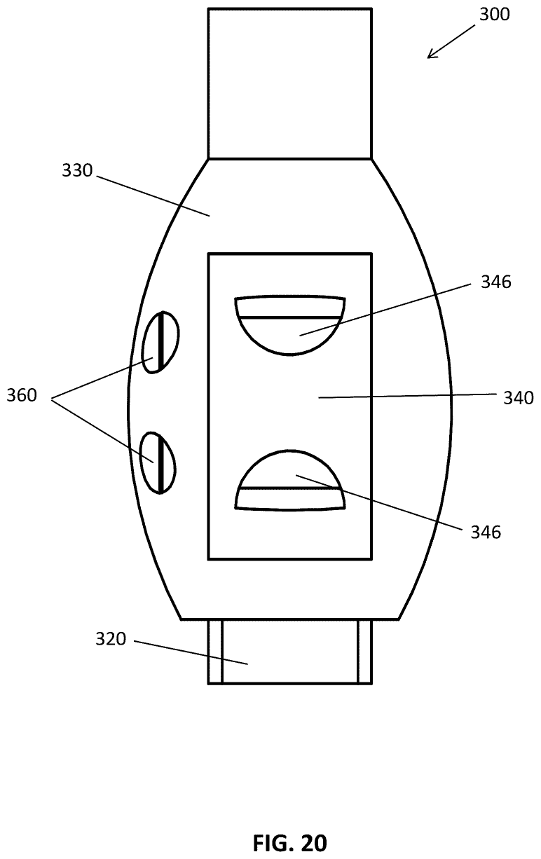

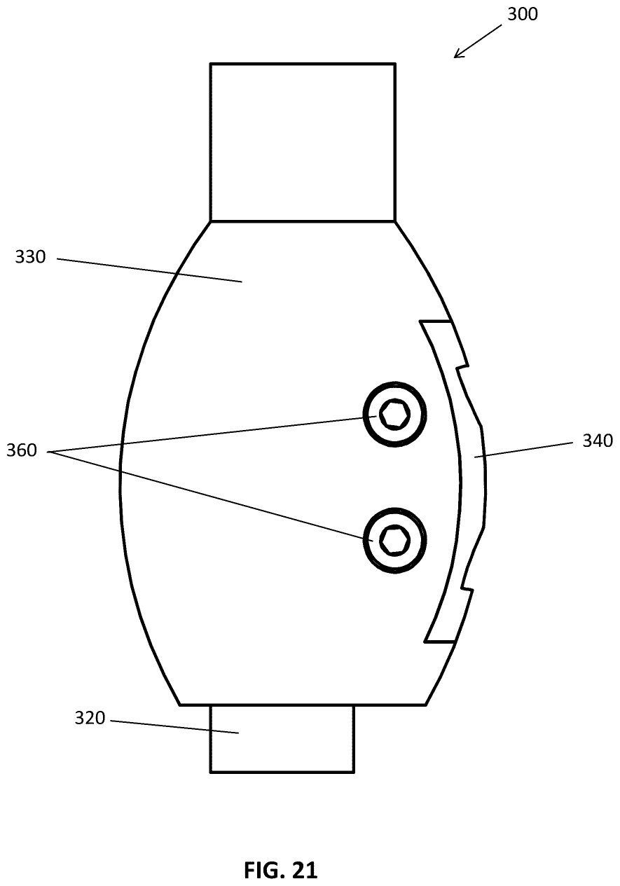

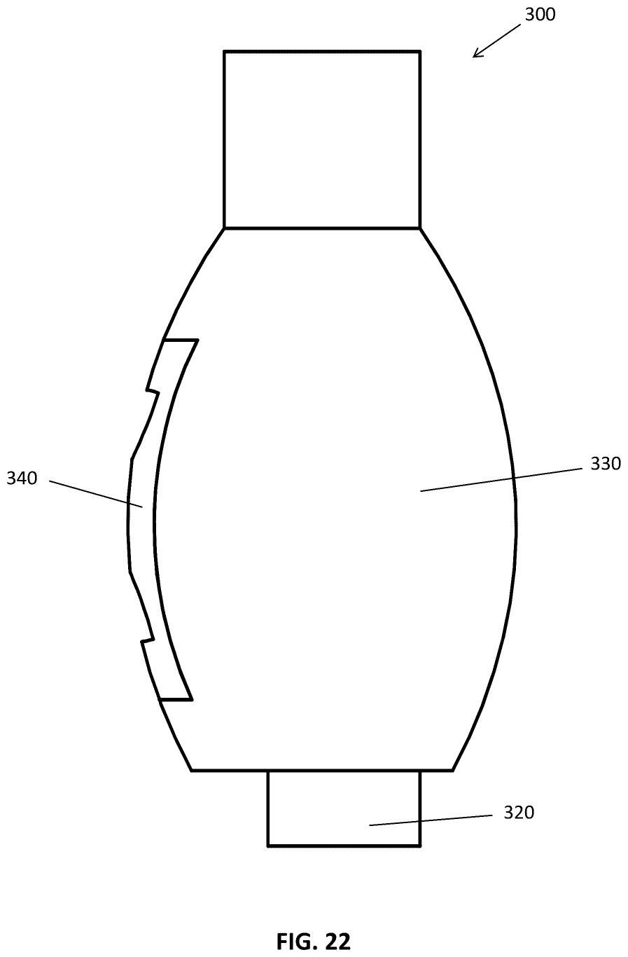

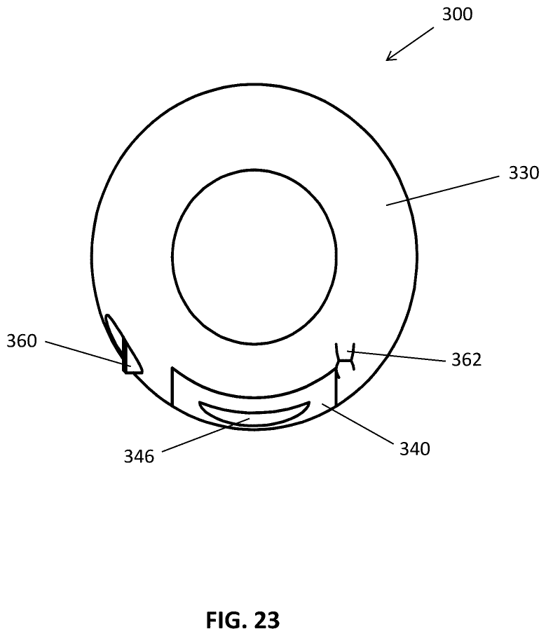

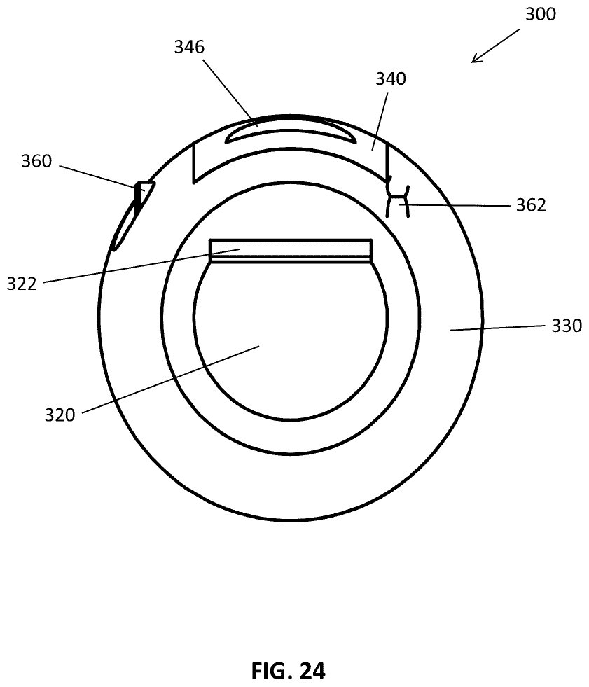

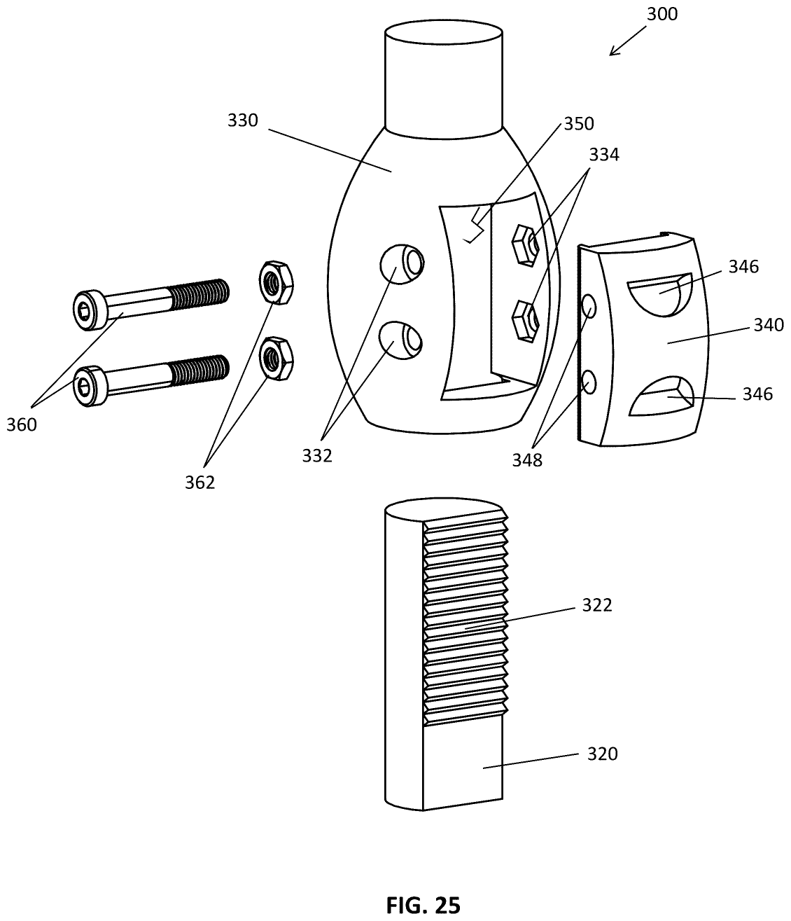

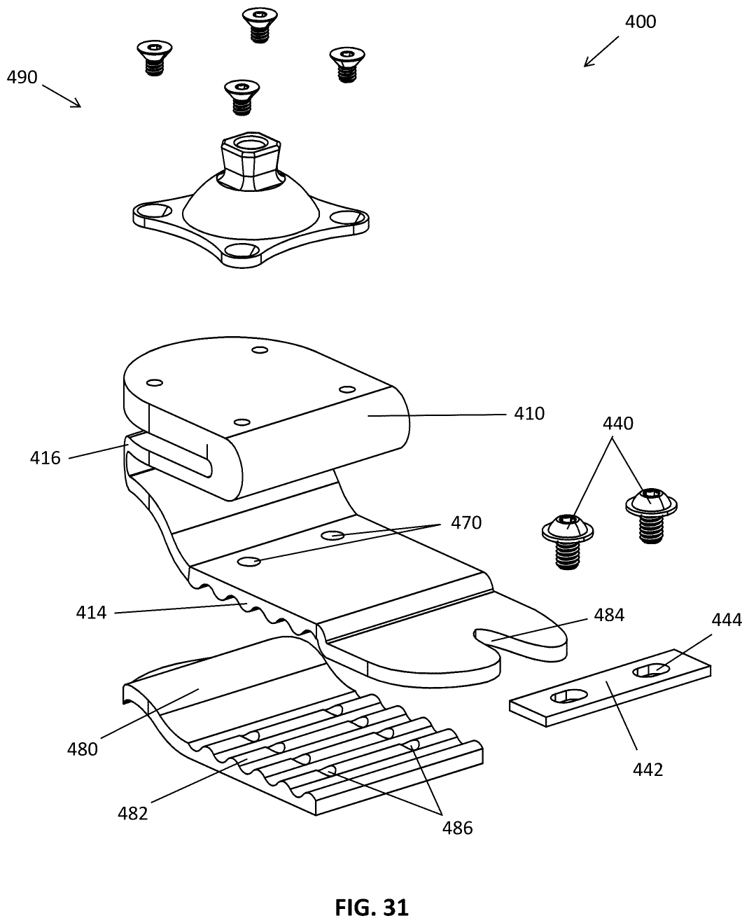

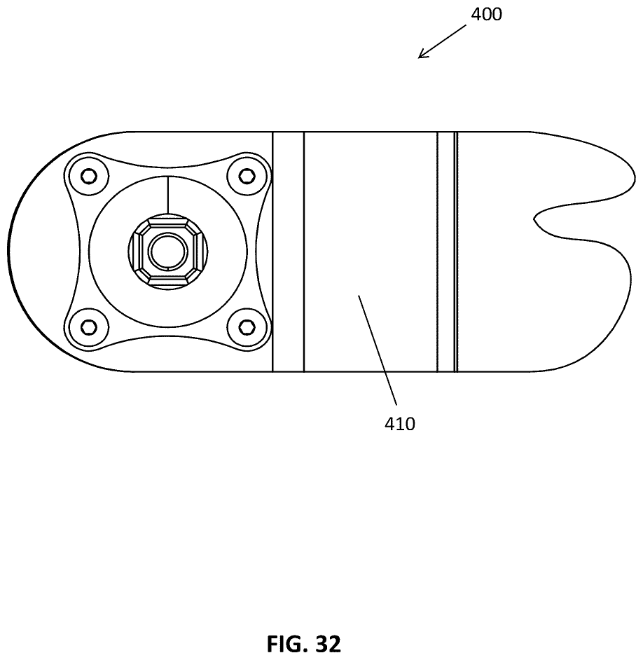

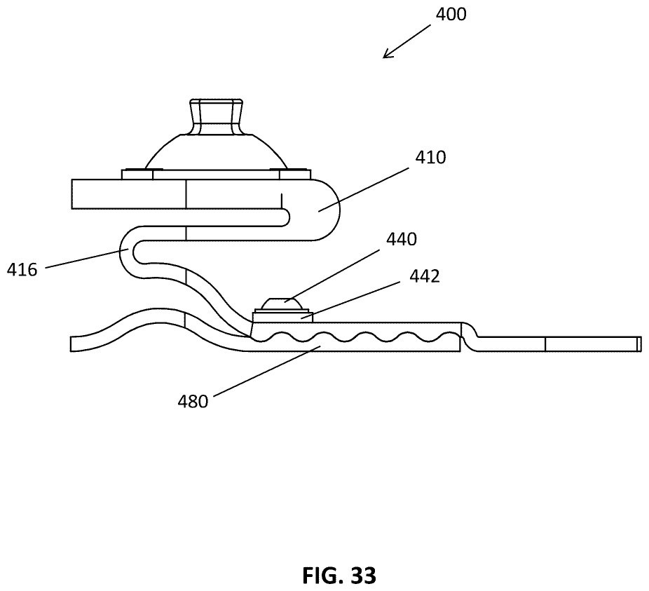

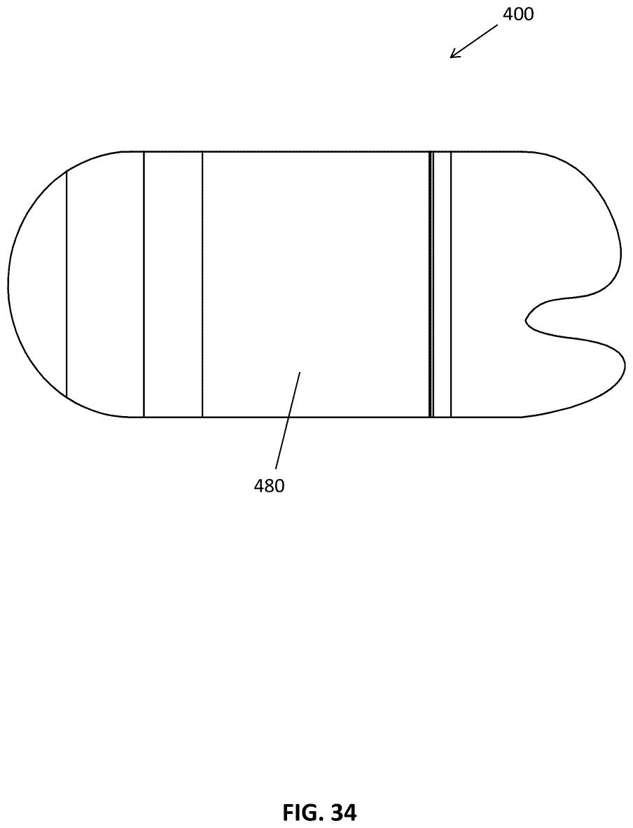

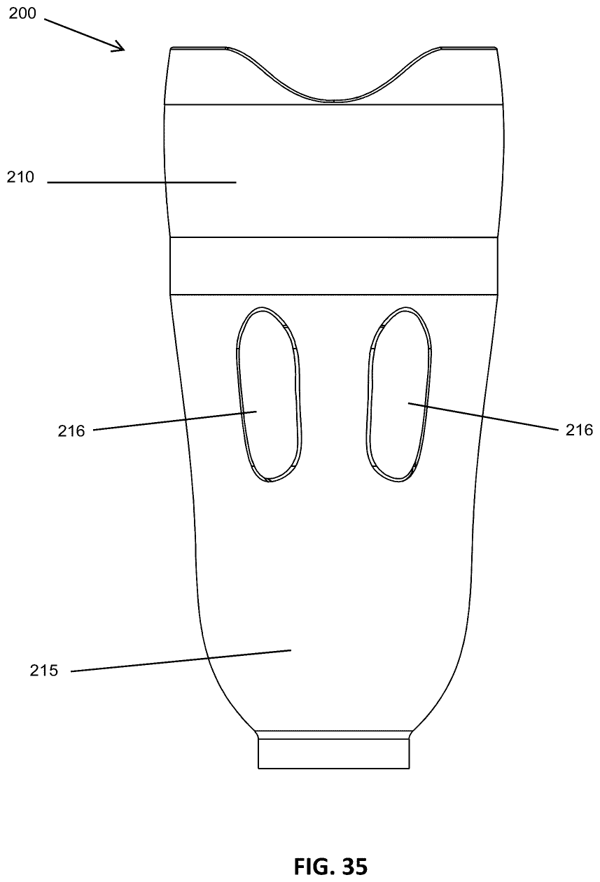

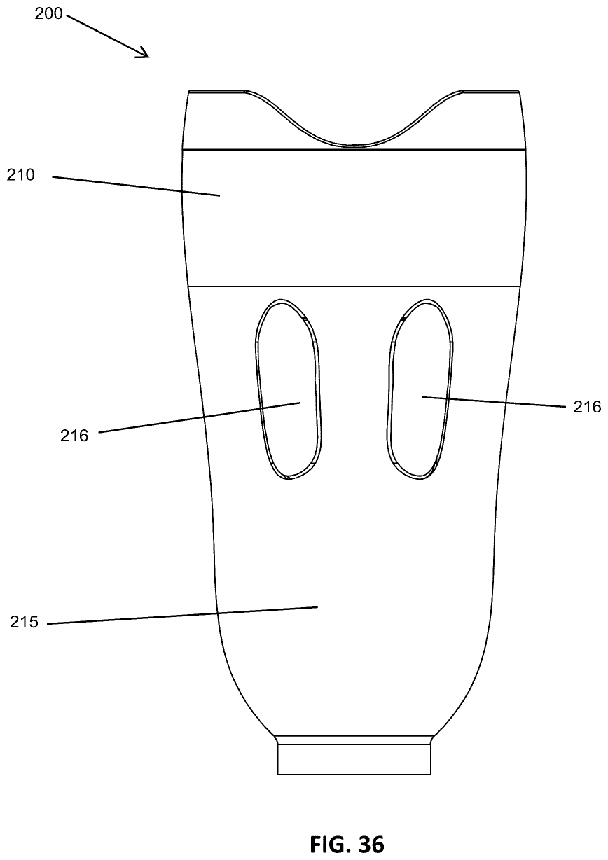

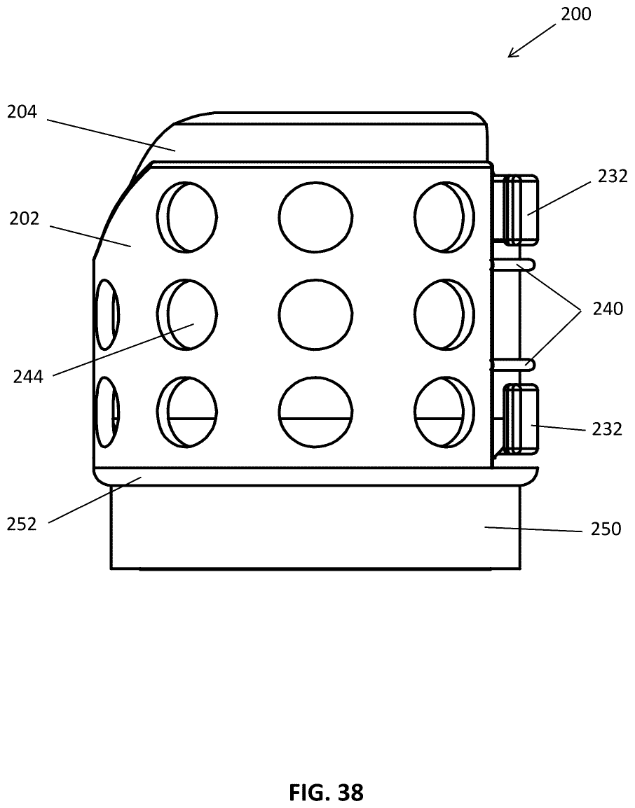

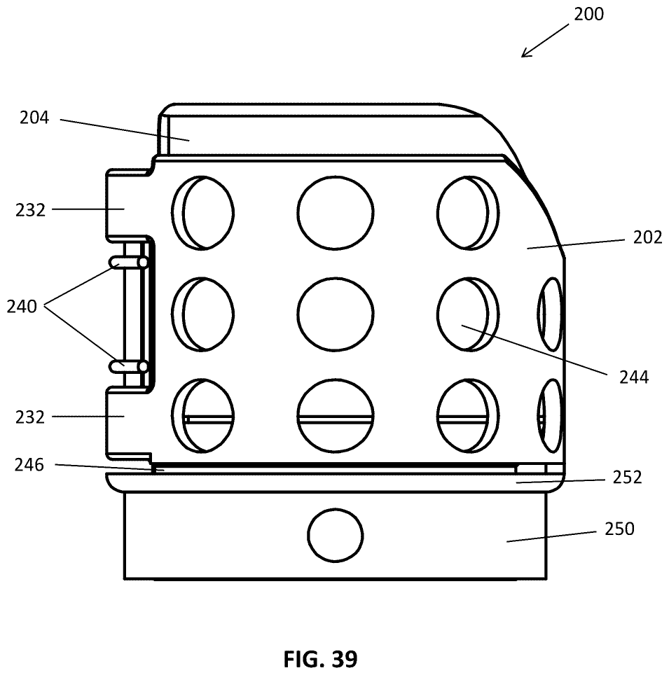

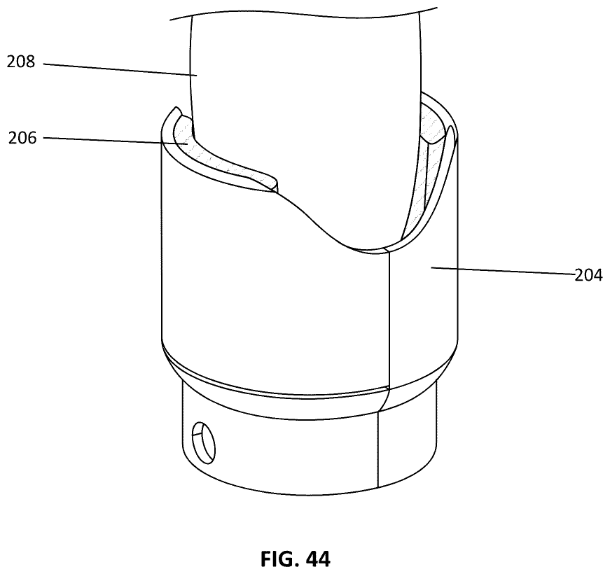

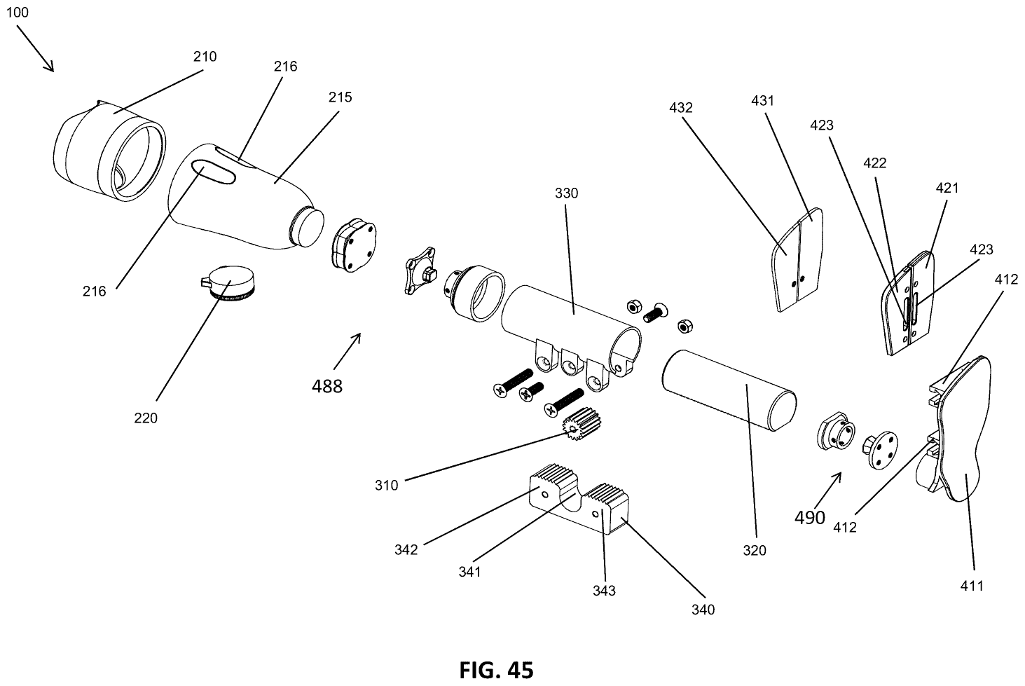

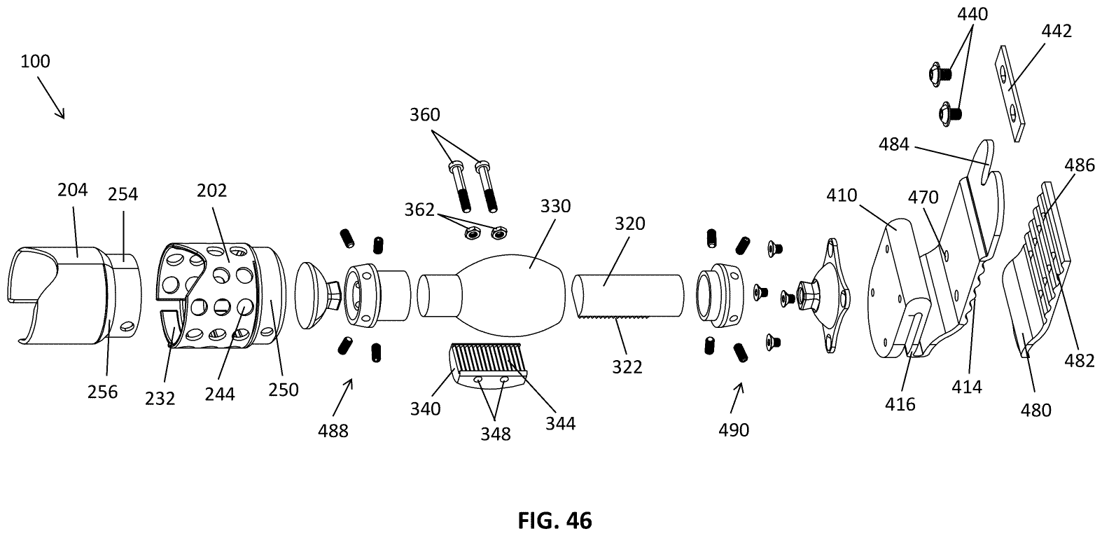

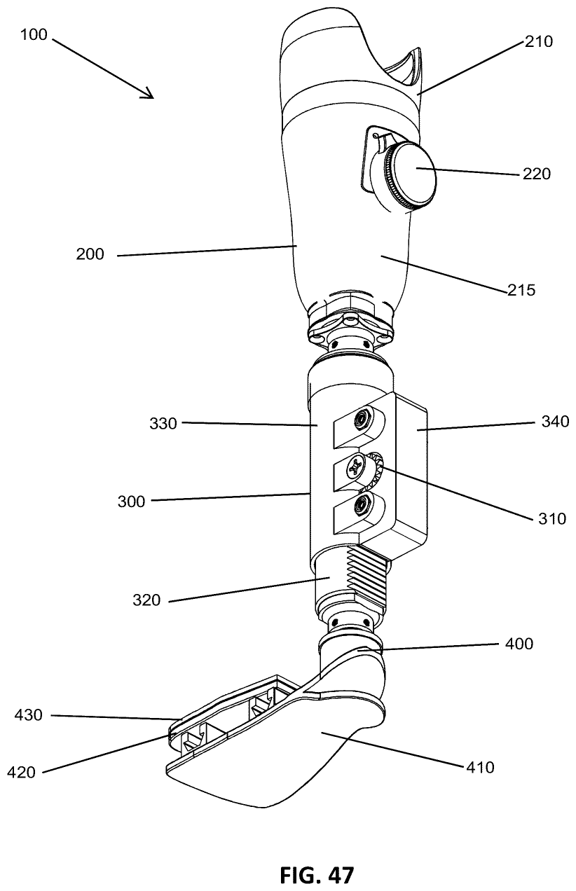

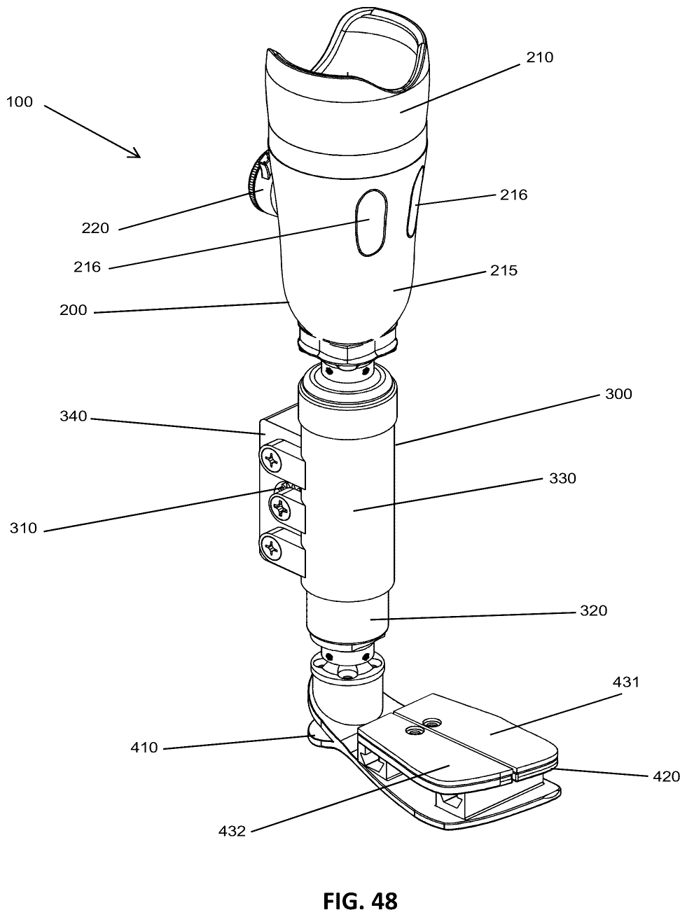

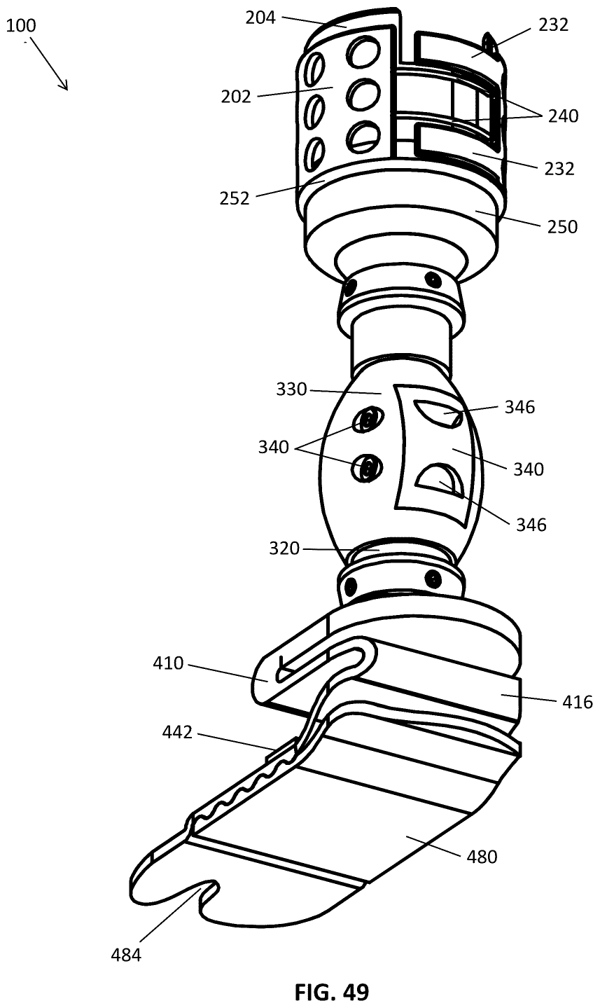

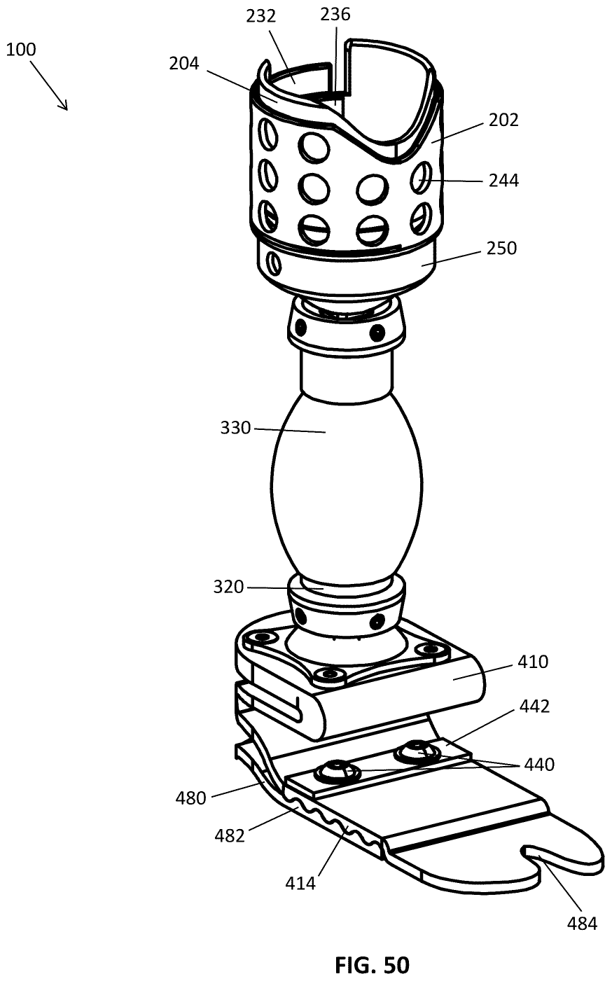

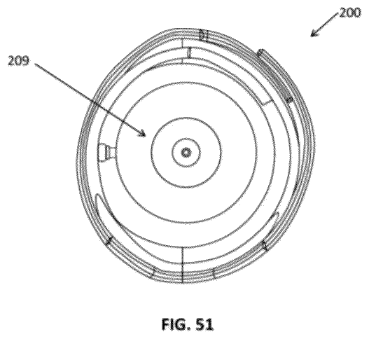

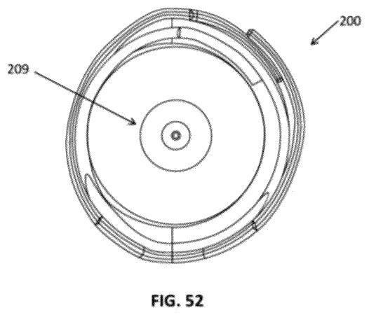

As shown in FIGS. 1-52, a prosthesis 100 is disclosed. In one embodiment, the prosthesis 100 may have three key components: the socket 200, pylon 300, and foot 400. Each of the socket 200, pylon 300, and foot 400 allows for its respective adjustability, including circumference of the residual limb, height of the limb, and/or width/length of the foot. Ideally, these principles would be altered in the future to accommodate for other lower limb differences, as well as upper limb differences.

### Socket 200

The socket 200 can be seen at least in FIGS. 1-11, 13, and 35-52. The socket 200 is the part of the prosthesis 100 that engages and securely holds the user's residual limb. In one embodiment the socket 200 comprises a top component 210, a bottom component 215, and a dial system 220 (also referred to as the dial 220). The top component 210 comprises a generally cylindrical section that surrounds the user's residual limb and holds the prosthesis 100 securely to the user's residual limb. The top component 210 has a first or upper end at or near the top edge of the generally cylindrical shape. The top component 210 also has a second or lower end opposite to the first or upper end at or near the bottom edge of the generally cylindrical shape. The top component 210 is intended to be removed and replaced, as needed, as the child or user grows. For example, this top component 210 may be replaced with a larger version that better fits the user as the user grows. To secure the top component 210, a secure attachment mechanism is utilized. The process of replacing the top component 210 should be performed in accordance with advice from a clinician.

The bottom component 215 comprises an elongated shape that mimics the shape of a human calf. The bottom component 215 has a first or upper end that connects securely to the second or lower end of the top component 210. The bottom component 215 also has a second or lower end that connects to the pylon 300. The second or lower end of the bottom component 215 may taper in diameter so as to further mimic the natural taper of a human calf. The dial 220 is attached to the bottom component 215 at the posterior window 217. The posterior window 217 comprises an opening or hole disposed within the bottom component 215 on the back side of the bottom component 215. The dial 220 adjusts through two anterior windows 216 and the posterior window 217. Other arrangements of windows within the bottom component 215 may be proposed by a clinician and formed in the bottom component 215 without departing from the scope of the disclosure.

The socket 200 may be produced with typical fabrication techniques performed in orthotics and prosthetics (O&P) clinics. In one embodiment, the shell of the socket 200 is made of materials such as carbon fiber, fiberglass, or epoxy/resin. The use of such materials is widely used and supported by O&P clinicians; however, other suitable materials may be substituted without departing from the scope of the disclosure. During fabrication, the socket 200 is split under the patellar bar with machinery in the clinic. The adaptability features of the dial 220 are then integrated into the socket 200 during its fabrication process.

Alternatively, the socket 200 may be created by modifying an existing fixed socket for a prosthesis. To create an adaptable socket 200 for use with a prosthesis 100, a trained clinician provided with an existing fixed socket having a patellar bar may divide the socket into a proximal segment 210 and a distal segment 215 just below the patellar bar. The proximal segment 210 and the distal segment 215 can then be connected to each other by affixing an attachment mechanism between the proximal segment 210 and the distal segment 215. A dial system 220 is then connected to the distal segment 215 to allow adjustments to the girth of the socket 200 by the end user. Such method allows the socket 200 to be adaptable to a user in leg girth and leg height. The modified socket 200 is adaptable to a user in leg girth via adjustment of the dial system 220. As the child or user grows, the proximal segment 210 of the socket 200 may be replaced with a larger proximal segment 210, thus allowing the socket 200 to be adaptable to a user in leg height.

Fine adjustments can be made to the fit of the socket 200 by rotating the dial 220 to increase or decrease the diameter of the top component 210. Greater adjustments can be achieved by replacing the top component 210 with another appropriately sized top component 210 when the child or user has outgrown the range of adjustments provided by rotation of the dial system 220 or to provide a taller top component 210 to compensate for the child's or user's increased height.

In another embodiment, the socket 200 comprises a lattice 202, a hardshell 204, padding 206, and a liner 208. The hardshell 204 is adjustable and the lattice 202 comprises an adjustable securement mechanism to allow for adjustability of the socket 200. Additionally, the socket 200 may be available in multiple lengths or sizes, to accommodate for the varying length of a child's or user's residual limb.

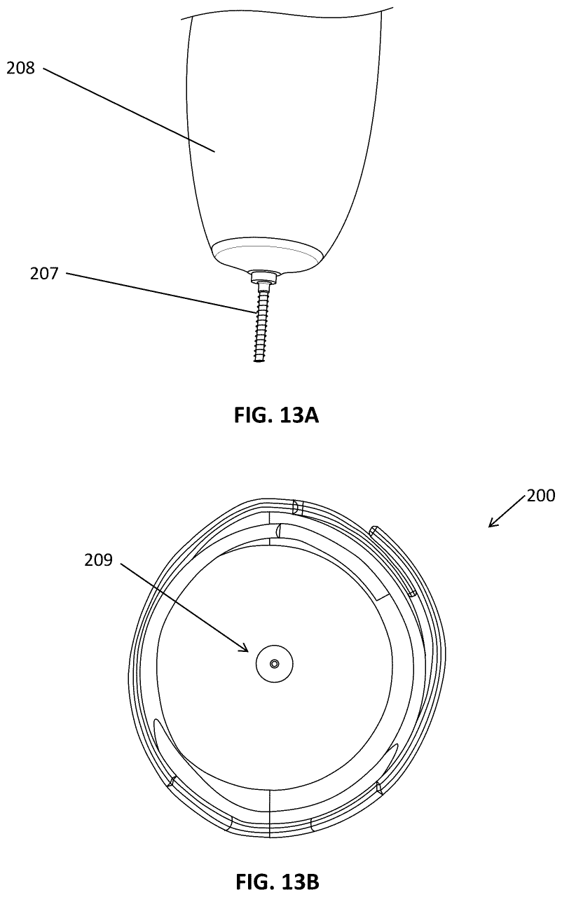

The liner 208 comprises a continuous layer of flexible material and a pin 207 attached to the distal end of the liner 208. The liner 208 may be made of a polymer, silicon, or a gel; however, other suitable materials may be substituted without departing from the scope of the disclosure. The material that the liner 208 is made of should be comfortable and allow for a snug fit on a user's residual limb. When the liner 208 is used by a user, the liner 208 is placed over the user's residual limb. The liner 208 is further attached to and detached from the hardshell 204 using a pin 207 and lock 209 liner suspension system. In this example, the lock 209 may be 55 millimeters in diameter and function using a ball bearing lock. However, any other mechanism, including but not limited to the mechanisms shown in FIGS. 13A, 13B, 51, and 52, for removably attaching the liner 208 to the hardshell 204 is hereby contemplated for use.

Padding 206 provides comfort and protection to the user's residual limb. More specifically, padding 206 is added to the hardshell 204, as needed, for relief of pressure intolerant areas and loading of pressure tolerant areas. Padding 206 may contain extra support pads to increase the level of padding inside the socket 200. The padding 206 comprises flexible material and may be made of a polymer, silicon, gel, cloth, or foam; however, other suitable materials may be substituted without departing from the scope of the disclosure. When padding 206 is used by a user, the user will place the padding 206 on top of the liner 208 and such padding 206 is held in place by friction or a soft hook-and-loop fastening material. However, any other mechanism for attaching the padding to the socket 200 is hereby contemplated for use.

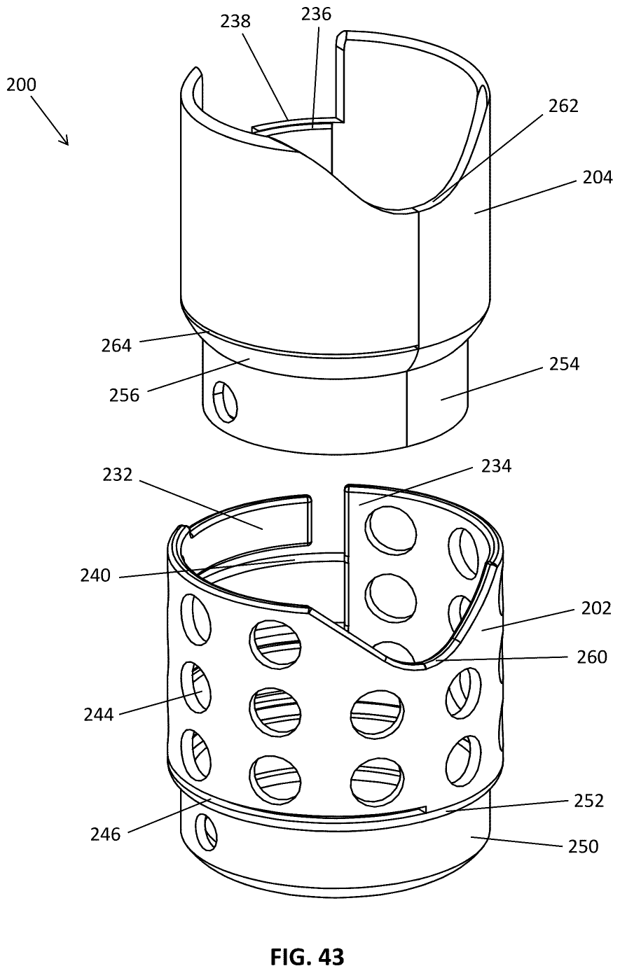

The hardshell 204 provides additional support and functionality to the user of the prosthesis 100. As one example, the hardshell 204 may comprise an outer flap 238 and an inner flap 236. The hardshell 204 may be made using injection molding and 3D printing along with material which is a polymer, plastic, or resin; however, other suitable materials may be substituted without departing from the scope of the disclosure. After a user has attached the liner 208 to the residual limb and removably attached padding 206 to the socket 200, the user may place the hardshell 204 around the liner 208 and/or padding 206. The inner flap 236 of the hardshell 204 is tucked inside of the outer flap 238 of the hardshell 204 so that the hardshell 204 is held in place around a user's residual limb. The outer flap 238 and the inner flap 236 of the hardshell 204 may attach to each other using a fastening mechanism 266 which may be any fastening mechanism, such as a hook and loop fastening mechanism, without departing from the scope of this disclosure. However, the fastening mechanism 266 should hold the hardshell 204 in place and provide adjustability, grip, and suspension of the user's residual limb inside the socket 200. By tucking the inner flap 236 inside of the outer flap 238 of the hardshell 204 and using the fastening mechanism 266, the volume of the hardshell 204 is adjustable to accommodate the user's residual limb size. Also, as illustrated in FIG. 43, the hardshell 204 may contain a gap 264 which extends approximately half of the circumference of the hardshell 204. The gap 264 allows and provides adjustability of the inner flap 236 while also maintaining the overall shape of the hardshell 204 when the hardshell 204 is adjusted to fit a user's residual limb. However, any other mechanism which allows for the adjustability of the hardshell 204 is hereby contemplated for use.

The hardshell 204 may further comprise a front recess 262 on the front or anterior side of the hardshell 204 to provide support and flexibility for the user's residual limb inside the socket 200. In particular, the front recess 262 may be configured to provide support, flexibility, and mobility for a user's knee when the user's residual limb is secured in the socket 200.

In this example, the hardshell 204 may further comprise a base 254, a shoulder 256, and an upper portion. The base 254 of the hardshell 204 may have a reduced diameter than the upper portion of the hardshell 204. The shoulder 256 of the hardshell 204 marks the change in diameter from the upper portion of the hardshell 204 to the base 254 of the hardshell 204. The change in diameter of the hardshell 204 allows the hardshell 204 to taper as the hardshell 204 extends towards the foot 400. The taper of the hardshell 204 is designed to mimic the natural taper of a human leg and allows for the appropriate fit of standard clothing. Further, any other mechanism which allows the tapering of the hardshell 204 is hereby contemplated for use.

The lattice 202 of the socket 200 comprises a layered, mesh-like structure designed to allow for adaptability in volume of the socket 200. The lattice 202 comprises flexible, yet rigid material and may be made of a polymer, silicon, gel, plastic, or resin; however, other suitable materials may be substituted without departing from the scope of the disclosure.

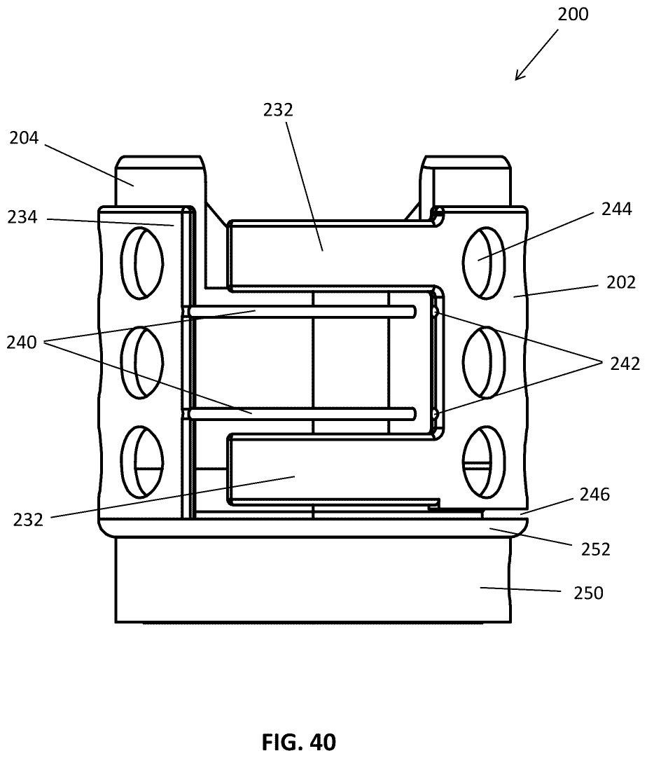

The lattice 202 comprises inner flaps 232, an outer flap 234, alignment guides 240, and alignment guide channels 242 to be used in adjusting the overall volume of the socket 200 to fit a user's residual limb. In this example, as seen in FIG. 40, two inner flaps 232 extend from an end of the lattice 202. From the opposite end of the lattice 202, two alignment guides 240 extend towards the inner flaps 232. When the lattice 202 is positioned around the hardshell 204, the alignment guides 240 are positioned in between the two inner flaps 232. Additionally, two alignment guide channels 242 extend through the interior of the lattice 202 and are designed to accept the alignment guides 240 to align the lattice 202 when the lattice 202 is in a compacted position. However, any other mechanism for providing adjustability of the socket 200 to a user or child's residual limb is hereby contemplated without departing from the scope of the disclosure.

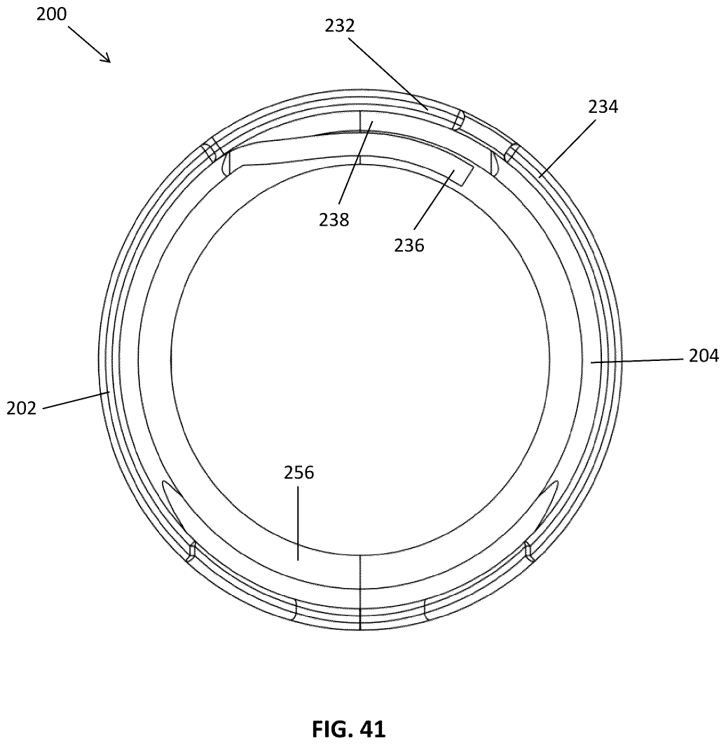

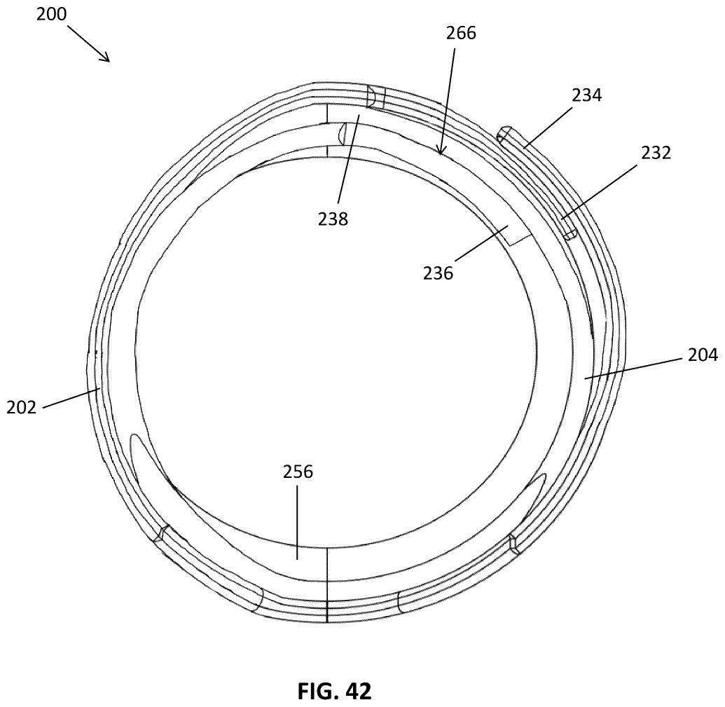

As seen in FIGS. 41 and 42, the lattice 202 can be adjusted in volume to fit the circumference of a user's residual limb. When the socket 200 is in a compacted position, the two inner flaps 232 of the lattice 202 are tucked inside of the outer flap 238 of the hardshell 204. As this occurs, the alignment guides 240 of the lattice 202 are directed inside of the alignment guide channels 242 which results in compaction of the lattice 202, hardshell 204, and socket 200. Additionally and/or alternatively, when the socket 200 is in a compacted position, the two inner flaps 232 of the lattice 202 may be tucked inside the outer flap 234 of the lattice 202. Expansion of the hardshell 204 and lattice 202 may be achieved by disengaging the outer flap 238 and the inner flap 236 of the hardshell 204, disengaging the alignment guides 240 from the alignment guide channels 242, and disengaging the inner flaps 232 of the lattice 202 from the outer flap 238 of the hardshell 204 and/or the outer flap 234 of the lattice 202.

In another example, the lattice 202 of the socket 200 may comprise a ratcheting system configured to secure and change the overall diameter of the lattice 202 and the socket 200. However, any other mechanism for adjusting the diameter of the socket 200 or adjustably securing the socket 200 is hereby contemplated for use. Other arrangements of adjustably securing the socket 200 to a user's residual limb may be contemplated or proposed by a clinician without departing from the scope of the disclosure.

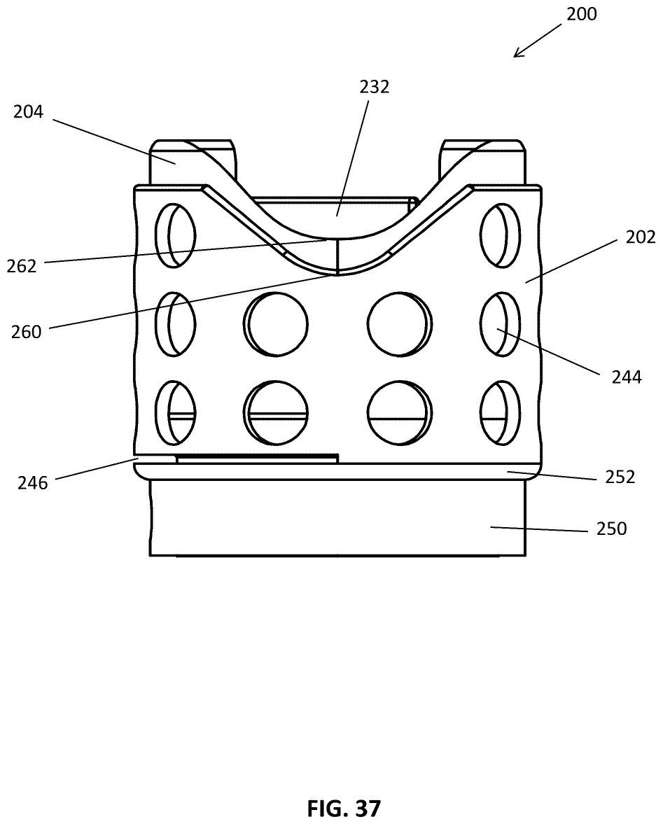

As illustrated in FIGS. 37 and 43, the lattice 202 may also contain a gap 246 which extends approximately half of the circumference of the lattice 202. The gap 246 allows and provides adjustability of the inner flaps 232 of the lattice 202 while also maintaining the overall shape of the lattice 202 when the volume of the lattice 202 is adjusted to fit a user's residual limb. Additionally, the lattice 202 further comprises a front recess 260 on the front or anterior side of the lattice 202 which provides support and relief for the user's patella inside the socket 200. In particular, the front recess 260 may be configured to provide support, flexibility, and mobility for a user's knee when the user's residual limb is secured in the socket 200.

As shown in FIG. 40 the lattice 202 may further comprise a base 250, a shoulder 252, and an upper portion of the lattice 202. The base 250 of the lattice 202 may have a reduced diameter than the upper portion of the lattice 202. The shoulder 252 of the lattice 202 marks the change in diameter from the upper portion of the lattice 202 to the base 250 of the lattice 202. The change in diameter of the lattice 202 allows the lattice 202 to taper as the lattice 202 extends towards the foot 400. The taper of the lattice 202 is designed to mimic the taper of a natural human leg and allows for the appropriate fit of standard clothing. Further, the base 250 and shoulder 252 of the lattice 202 are designed to fit snugly around the base 254 and shoulder 256 of the hardshell 204. Further, any other mechanism which allows the tapering of the lattice 202 is hereby contemplated for use.

The lattice 202 may be custom designed by a user with a variety of different colors and/or patterns. In this example, lattice 202 comprises a plurality of openings 244. The lattice 202 may be manufactured using 3D printing or any other advanced fabrication techniques. The mesh-like design of the lattice 202, allowed by the plurality of openings 244, provides an overall lighter weight, greater flexibility, increased breathability, and improved comfort and impact absorption to the user of the prosthesis 100.

The materials of the socket 200 are sufficiently rigid for automatic accommodation and compression of the residual limb. The socket 200 is successfully adjusted after confirming the final securement mechanism. Fine adjustments can be made to the fit of the socket 200 by adjusting the applicable securement mechanism to increase or decrease the diameter of the socket 200. Greater adjustments can be achieved by replacing the socket 200 with another appropriately sized socket 200 when the child or user has outgrown the range of adjustments provided by adjustment of the lattice 202 and hardshell 204 or to provide a taller socket 200 to compensate for the child's or user's increased height. Other arrangements of adjustably securing the socket 200 to a user's residual limb may be contemplated or proposed by a clinician without departing from the scope of the disclosure.

### Pylon 300

The pylon 300 can be seen at least in FIGS. 1-4, 6-10, 14-25 and 45-50. The pylon 300 is disposed in between and securely attached to the socket 200 and the foot 400. The pylon 300 provides for vertical adjustability of the prosthesis 100, allowing height adjustment as the child or user becomes taller.

The pylon 300 is comprised of a guiderail 330. In one embodiment, guiderail 330 comprises a generally cylindrical hollow tube having an inner diameter and an outer diameter. In another embodiment, the guiderail 330 has a convex or a barrel-shaped profile. At a first or upper end of the pylon 300, the first or upper end of the guiderail 330 operably attaches to the lower end of the socket 200 using a first connection mechanism 488. Any connection mechanism is hereby contemplated for use, including but not limited to, using one or more fasteners to securely fasten two or more platforms together where one platform is connected to the lower end of the socket 200 and at least a second platform is connected to the upper end of the guiderail 330.

A rack 320 inserts into the guiderail 330 and is movable within the guiderail 330. The rack 320 may comprise an elongated generally cylindrical or an elongated generally rectangular solid. The cylinder or rectangular prism forming the rack 320 may have a flattened portion running from end to end, and a series of teeth suitable for engaging a gear, such as pinion 310 or a lockrack engaging surface 344, are formed on the flattened side of the cylinder or rectangular prism. The rack 320 has an outer diameter or perimeter that is approximately equal to or slightly less than the inner diameter or perimeter of the guiderail 330 such that the rack 320 fits snugly inside the guiderail 330. The position of the rack 320 can be moved up and down within the guiderail 330 to provide adjustable height to the prosthesis 100. The lower end of the rack 320 attaches securely to the foot 400. In one embodiment, an attachment member 350 may connect the guiderail 330 to the rack 320, so that the guiderail 330 and rack 320 can be held together when a lockrack 340 is disengaged. The attachment member 350 may be made of a loaded or unloaded clastic material or be constructed by another mechanical system suited to hold two free objects together. However, any other mechanism is hereby contemplated for use.

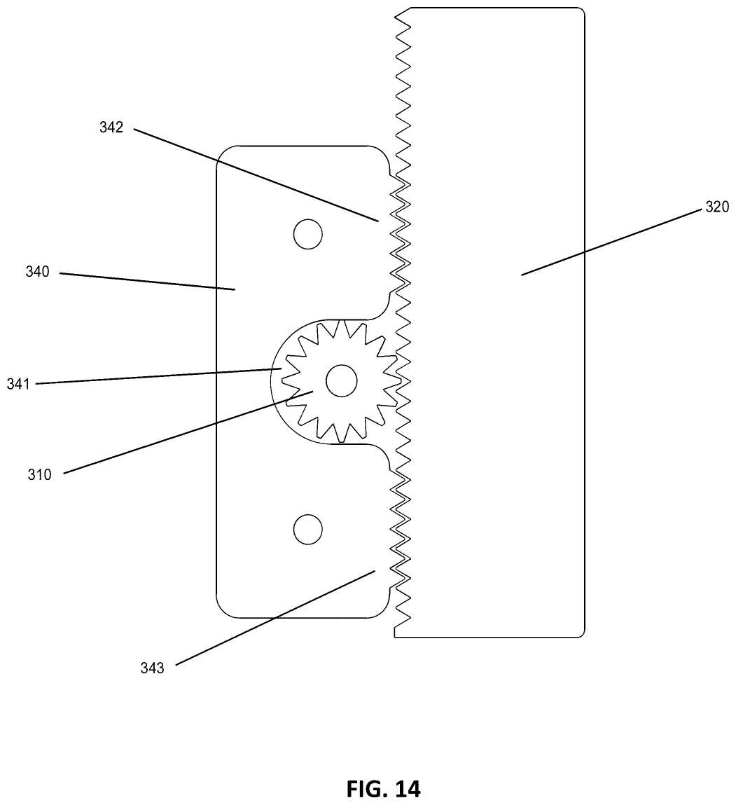

In one embodiment, a pinion 310 engages the rack 320. The pinion 310 comprises a generally circular gear having teeth that engage the teeth of the rack 320. Rotation of the pinion 310 creates linear movement in the rack 320, achieving height adjustment of the prosthesis 100. A locking mechanism 340, also called a lockrack 340, is fixedly attached to the guiderail 330. As shown in FIG. 14, the lockrack 340 comprises a generally rectangular block with a notch 341 formed in the approximate center of the lockrack 340 to accommodate the pinion 310. Formation of the notch 341 in the lockrack 340 on the side of the lockrack 340 that engages the rack 320 creates a first rack engaging surface 342 on one side of and above the notch 341 and a second rack engaging surface 343 on the other side of and below the notch 341. The first rack engaging surface 342 and second rack engaging surface 343 have teeth complimentary to the teeth of the rack 320. The notch 341 allows the locking mechanism 340 to surround and cover the pinion 310, preventing the interface of the pinion 310 and the rack 320 from coming into contact with objects such as fingers and clothing.

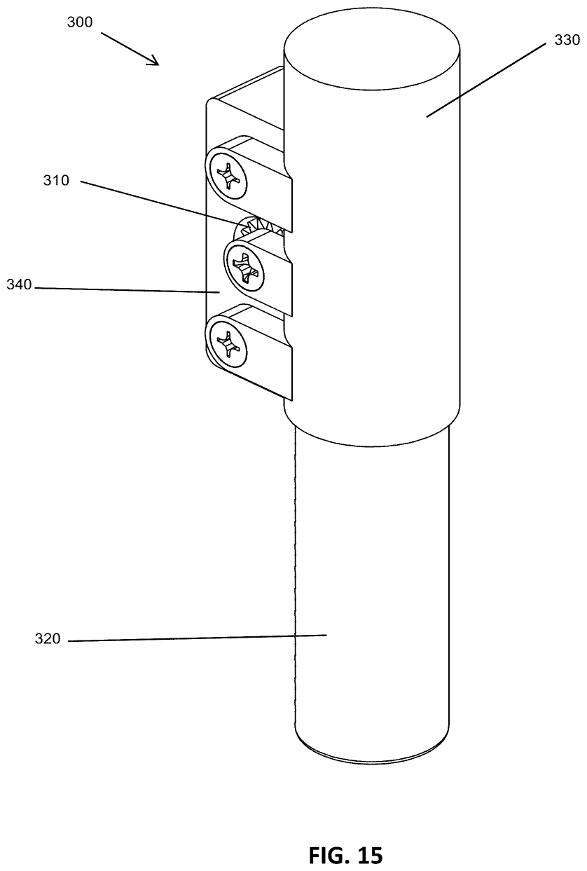

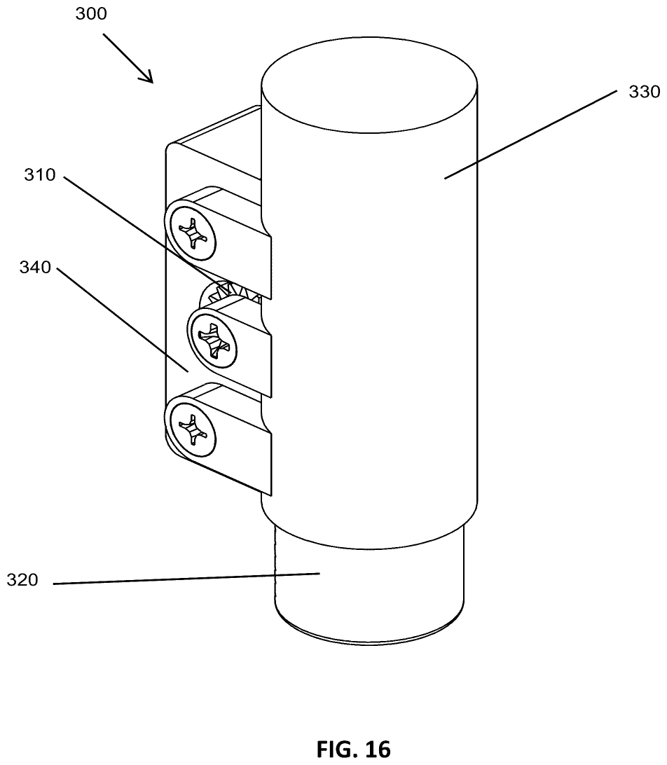

As can be seen most easily in FIGS. 15 and 16, in one embodiment, the height of the pylon 300 is adjusted via rotation of the pinion 310 which actuates the rack 320 downwards, increasing the height of the pylon 300. To prevent the rack 320 from adjusting when loaded, the lockrack 340 holds the rack 320 in place, using complimentary teeth that fit into the teeth of the rack 320. The lockrack 340 is held securely in place against the rack 320 to prevent it from adjusting in height during use of the prosthesis 100. In one embodiment, the lockrack 340 is secured to the guiderail 330 using two nuts 362 and two bolts 360; however, any suitable attachment mechanism may be used to securely hold the lockrack 340 to the guiderail 330. Further, any other mechanism for pylon 300 height adjustment is hereby contemplated for use. In one embodiment, the pylon 300 can extend from four inches to seven inches in height; however, other height ranges may be used for pylon 300 without departing from the scope of the disclosure. In FIG. 15, the pylon 300 is depicted with the rack 320 in its fully extended position. In FIG. 16, the pylon 300 is shown with the rack 320 in its fully retracted or fully closed position.

In another embodiment, a lockrack 340 engages the rack 320. The rack 320 may have a rack engaging surface 322 having teeth. The rack 320 may have an outer diameter that is approximately equal to or slightly smaller than the inner diameter of the guiderail 330, ensuring a snug fit. The lockrack 340 comprises a lockrack engaging surface 344 having teeth that engage the teeth of the rack engaging surface 320. The lockrack 340 further comprises one or more grips 346 that allow a user to remove and insert the lockrack 340 to and from the guiderail 330. Placement of the lockrack 340 within an opening in a posterior or back side of the guiderail 330 permits the lockrack 340 to secure the rack 320 in a desired position. The lockrack 340 may have an outer surface that is generally convex in shape and designed to be flush with the outer surface of the guiderail 330 when engaged with the rack 320. The lockrack 340 may be designed to be concealed within the guiderail 330 or attached to the exterior of the guiderail 330.

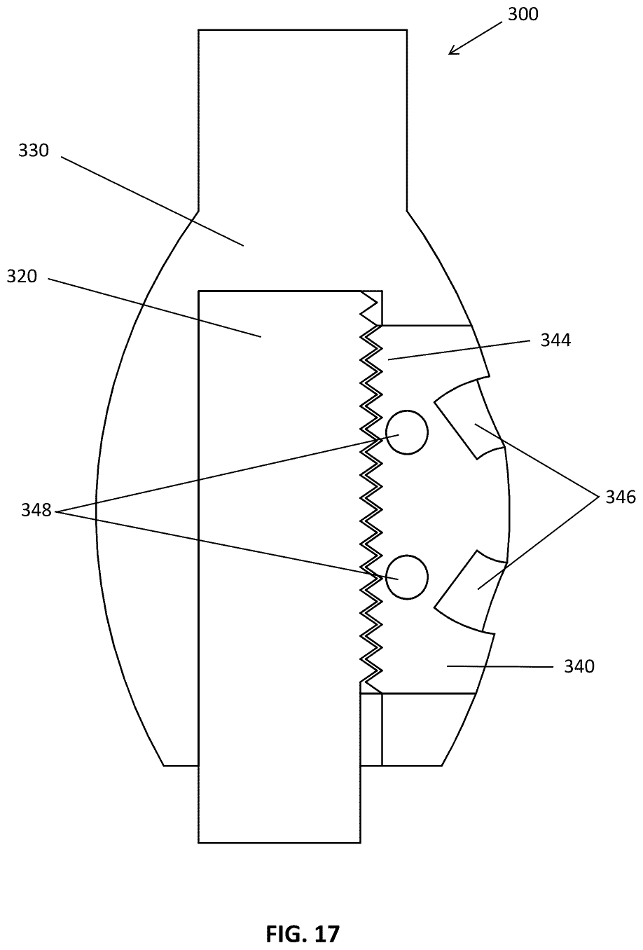

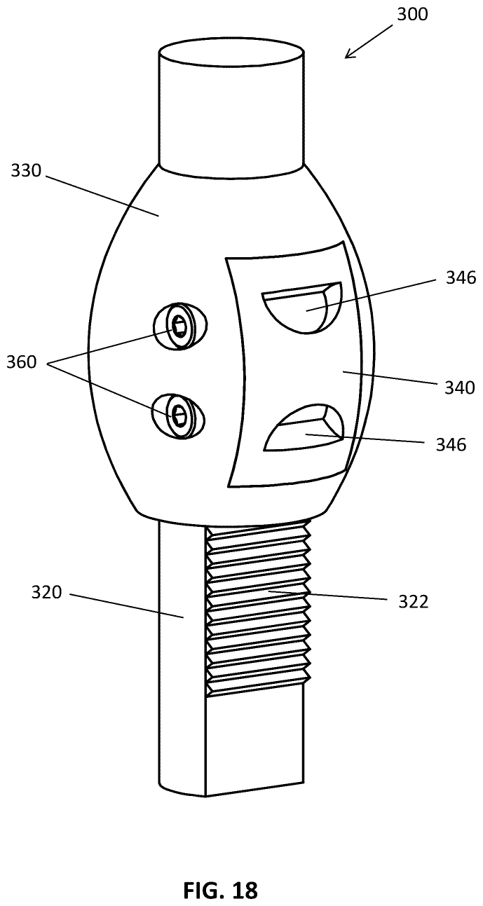

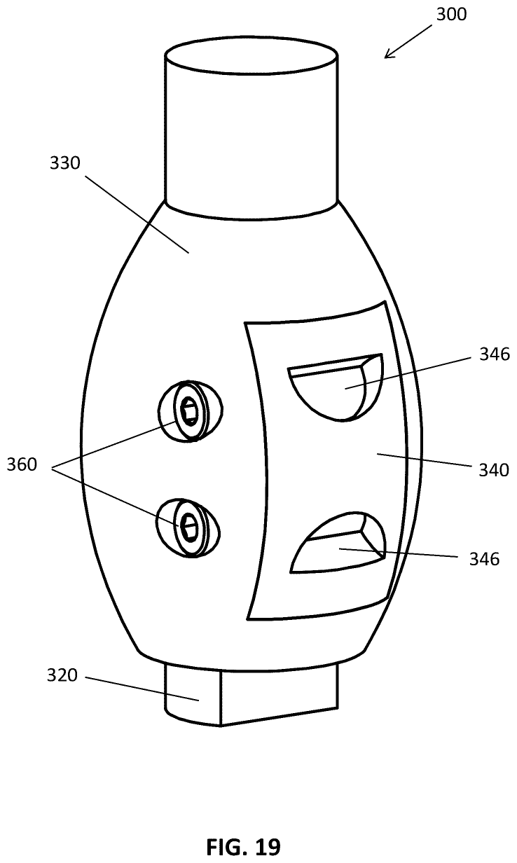

As can be seen most easily in FIGS. 17-19, the height of the pylon 300 can be adjusted via removal of the lockrack 340 from the guiderail 330 which allows free movement of the rack 320 downwards, increasing the height of the pylon 300. To prevent the rack 320 from adjusting when loaded, the lockrack 340 holds the rack 320 in place, using complimentary teeth that fit into the teeth of the rack 320. The lockrack 340 is held securely in place against the rack 320 to prevent it from adjusting in height during use of the prosthesis 100. In one embodiment, the lockrack 340 is secured to the guiderail 330 using two bolts 360 and/or two nuts 362; however, any suitable attachment mechanism may be used to securely hold the lockrack 340 to the guiderail 330. The bolts 360 are inserted into a bolt head recess 332 of the guiderail 330 and through a bolt opening 348 in the lockrack 340. The bolts 360 are secured with two nuts 362 that are located in a nut opening 334 of the guiderail 330. Further, any other mechanism for pylon 300 height adjustment is hereby contemplated for use. In one embodiment, the pylon 300 can extend up to two inches in height from the shortest setting; however, other height ranges may be used for pylon 300 without departing from the scope of the disclosure. In FIG. 19, the pylon 300 is depicted with the rack 320 in its fully retracted or fully closed position. In FIG. 18, the pylon 300 is shown with the rack 320 in its fully extended position.

The guiderail 330 of pylon 300 may be fabricated using metals (stainless steel, aluminum, and titanium alloys) or other suitable materials such as carbon fiber composites. The rack 320, pinion 310, and lockrack 340, as well as any attachment hardware, can be machined from metal. Due to the complexity and geometry of the guiderail 330, methods involving 3D printing with carbon fiber/carbon fiber reinforced filament may also be used to fabricate the guiderail 330.

### Foot 400

The foot 400 can be seen at least in FIGS. 1-12, 26-34, and 45-50. The foot 400 provides an adjustable stable base for the prosthesis 100. The foot 400 mimics a natural human foot shape and allows the user to naturally perform activities such as standing and walking. The foot 400 operably attaches to the prosthesis 100 at a lower end of the pylon 300 using a second connection mechanism 490. Any connection mechanism is hereby contemplated for use, including but not limited to, using one or more fasteners to securely fasten two or more platforms together where one platform is connected to the lower end of the pylon 300 and at least a second platform is connected to the upper end of the foot 400. In one embodiment, the foot 400 comprises three sections: a base 410, a width-expansion layer 420, and a length-expansion layer 430.

The base 410 consists of a split shank heel 411, inspired by a natural foot shape, with two slotted tracks 412 attached to the anterior of the base 410 for attachment of the width-expansion layer 420.

The width-expansion layer 420, which may alternatively be referred to as the width-expansion platform 420 without departing from the scope of the disclosure, allows for adjustability of the width of the foot 400. The width-expansion platform 420 comprises a first width-expansion plate 421 and a second width-expansion plate 422 positioned on top of the slotted tracks 412 on the anterior of the foot 400. The first width-expansion plate 421 and the second width-expansion plate 422 have differing shapes to mimic the shape of a person's toes. The first width-expansion plate 421 and the second width-expansion plate 422 each have two holes that align with the tracks 412 of the base 410 to secure them at a variety of widths using screws and bolts. The first width-expansion plate 421 and the second width-expansion plate 422 also each have a slot 423 that enables connection of the length-expansion layer 430. The fasteners securing the first width-expansion plate 421 to the tracks 412 and the second width-expansion plate 422 to the tracks 412 can be loosened, allowing the first width-expansion plate 421 and second width-expansion plate 422 to be moved from side to side to adjust for growth of the child's or user's foot, and tightened to keep the width-expansion layer 420 stationary.

The length-expansion layer 430, which may alternatively be referred to as the length-expansion platform 430 without departing from the scope of the disclosure, allows for adjustability of the length of the foot 400. The length-expansion layer 430 is positioned on top of the width-expansion layer 420. The length-expansion layer 430 comprises a first length-expansion plate 431 and a second length-expansion plate 432. The first length-expansion plate 431 and the second length-expansion plate 432 have differing shapes to mimic the shape of a person's toes, where the first length-expansion plate 431 has a similar size and shape to the first width-expansion plate 421, and the second length-expansion plate 432 has a similar size and shape to the second width-expansion plate 422. The first length-expansion plate 431 contains at least one hole for a bolt to pass through and secure the first length-expansion plate 431 to the slot 423 in the first width-expansion plate 421. Similarly, the second length-expansion plate 432 contains at least one hole for a bolt to pass through and secure the second length-expansion plate 432 to the slot 423 in the second width-expansion plate 422. The bolts securing the first length-expansion plate 431 to the first width-expansion plate 421 and the second length-expansion plate 432 to the second width-expansion plate 422 can be loosened, allowing the first length-expansion plate 431 and second length-expansion plate 432 to be moved forward and back to adjust for growth of the child's or user's foot, and tightened to keep the length-expansion layer 430 stationary.

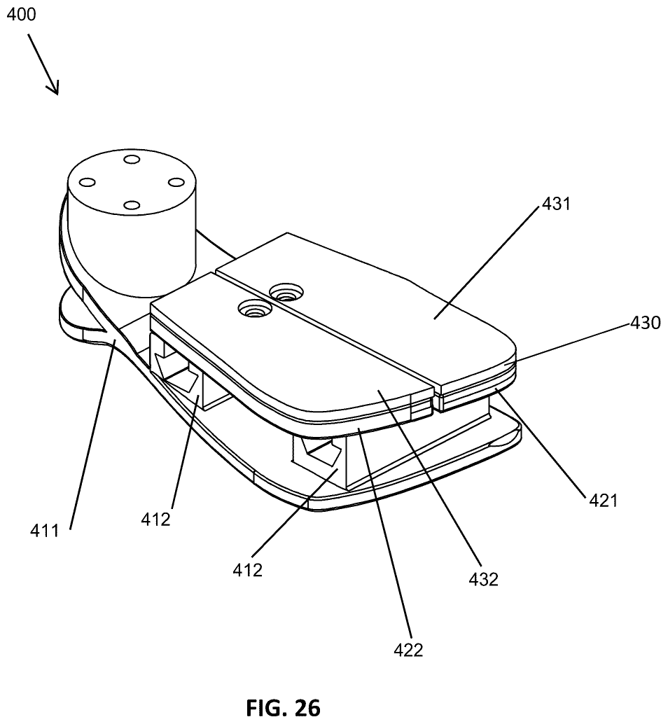

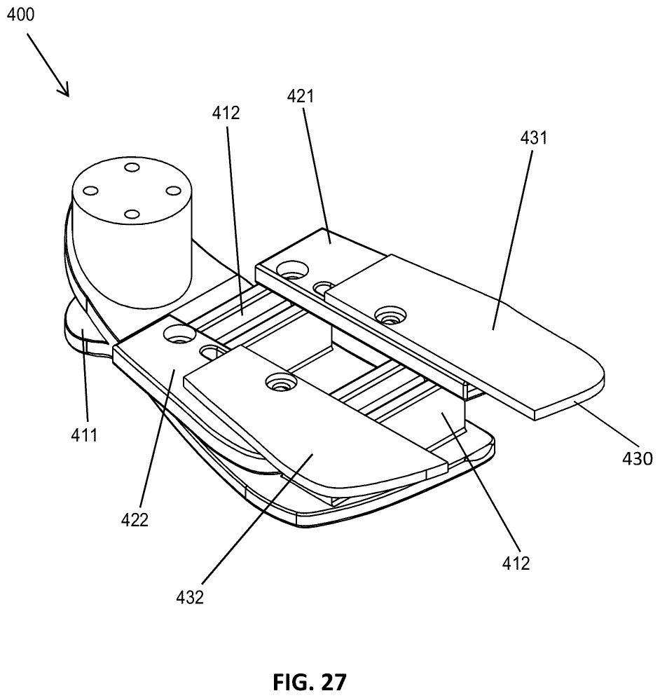

As can be seen most easily in FIGS. 26 and 27, the use of the described width-expansion layer 420 and length-expansion layer 430 allows adjustment of the length and width of the foot 400 as the child or user grows. FIG. 26 shows the foot 400 with length and width fully retracted or fully closed. FIG. 27 shows the foot 400 with length and width fully expanded. In one embodiment, the described design of the foot 400 allows for 0.5 inches of width expansion and 0.8 inches of width expansion; however, other expansion ranges may be used without departing from the scope of the disclosure.

In another embodiment, the foot 400 comprises three sections: a base 410, a medial expansion plate 450, and a lateral expansion plate 460. The base 410 consists of a split shank heel 411, inspired by a natural foot shape and a split keel with a plurality of slots 470 extending through the base 410 for attachment of a medial expansion plate 450 and a lateral expansion plate 460.

The medial expansion plate 450, which may alternatively be referred to as the medial expansion platform 450 without departing from the scope of the disclosure, allows for adjustability of the width and length of the foot 400. The medial expansion platform 450 is positioned anteriorly to the split keel of the base 410. The medial expansion platform 450 can be extended diagonally to adjust the width and length of the foot 400. The shape of the medial expansion platform 450 mimics the shape of a person's toes. The medial expansion platform 450 has at least one hole for at least one fastener 440 to pass through. The at least one hole of the medial expansion platform 450 aligns with the plurality of slots 470 of the base 410 to secure the medial expansion platform 450 at a variety of widths and lengths using a common fastener 440, such as screws or bolts. However, any other mechanism of fastening the medial expansion platform 450 to the base 410 is hereby contemplated for use. The slots 470 in the base 410 are incremented to correspond with a child's or user's shoe size to allow for proper fit in children's shoes as well as for adjustability so that the medial expansion platform 450 can be adjusted as a child or user grows. The fastener 440 securing the medial expansion platform 450 to the slots 470 can be loosened, allowing the medial expansion platform 450 to be adjusted diagonally to accommodate for change in size of the child's or user's foot, and tightened to keep the medial expansion platform 450 stationary. However, any other mechanism for foot 400 size adjustment is hereby contemplated for use.

The lateral expansion plate 460, which may alternatively be referred to as the lateral expansion platform 460 without departing from the scope of the disclosure, allows for adjustability of the length of the foot 400. The lateral expansion platform 460 is positioned anteriorly to the split keel of the base 410 and next to the medial expansion platform 450. The lateral expansion platform 460 can be extended anteriorly to adjust the length of the foot 400. The shape of the lateral expansion platform 460 mimics the shape of a person's toes. The lateral expansion platform 460 has at least one hole for at least one fastener 440 to pass through. The at least one hole of the lateral expansion platform 460 aligns with the plurality of slots 470 of the base 410 to secure the lateral expansion platform 460 at a variety of lengths using a common fastener 440, such as screws or bolts. However, any other mechanism of fastening the lateral expansion platform 460 to the base 410 is hereby contemplated for use. The slots 470 in the base 410 are incremented to correspond with a child's or user's shoe size to allow for proper fit in children's shoes as well as for adjustability so that the lateral expansion platform 460 can be adjusted as a child or user grows. The fastener 440 securing the lateral expansion platform 460 to the slots 470 can be loosened, allowing the lateral expansion platform 460 to be adjusted anteriorly to adjust for change in size of the child's or user's foot, and tightened to keep the lateral expansion platform 460 stationary. However, any other mechanism for foot 400 size adjustment is hereby contemplated for use.

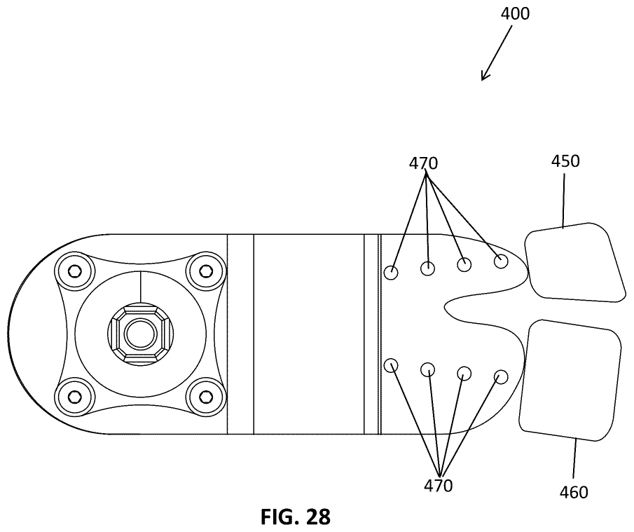

As can be seen most easily in FIG. 28, the described medial expansion plate 450 and lateral expansion plate 460 allow for adjustment of the length and width of the foot 400 as the child or user grows. The foot 400 may be fabricated using methods of 3D printing with carbon fiber-reinforced filament or other suitable materials or methods. The components of the foot 400 may be fastened together with standard fasteners 440 such as metric screws and bolts. The foot 400 may further comprise a thin overlapping plastic layer between components to avoid debris, along with a foot shell and mesh sock to mimic the shape and function of a child's or user's foot.

In another embodiment, the foot 400 comprises two sections: a base 410 and a heel expansion platform 480. The base 410 consists of an upper portion 416 comprising a serpentine bend to mimic a natural ankle and the top portion of a split shank heel 411, inspired by a natural foot shape. The base 410 comprises two slots 470 extending through the base 410 for attachment of a heel expansion platform 480. The base 410 further comprises a toe notch 484 designed to mimic the toes of a natural human foot and allow for donning of different forms of shoe wear. Shoe wear can include, but is not limited to, sandals, flip flops, running shoes, tennis shoes, and/or dress shoes.

The heel expansion platform 480 allows for adjustability of the length of the foot 400. The heel expansion platform 480 is positioned beneath the base 410, comprising the lower portion of the split shank heel 411. The heel expansion platform 480 can be extended behind the base 410 to adjust the length of the foot 400. The shape of the heel expansion platform 480 mimics the shape of a human's natural heel. The heel expansion platform 480 has two rows of a plurality of slots 486 for at least one fastener 440 to pass through. The two rows of a plurality of slots 486 of the heel expansion platform 480 align with the plurality of slots 470 of the base 410 to secure the heel expansion platform 480 at a variety of different positions corresponding to different lengths of foot 400 using a common fastener 440, such as screws or bolts. In the middle portion of the heel expansion platform 480 is an oscillating surface 482 which corresponds with an oscillating thickness of the heel expansion platform 480. The oscillating surface 482 oscillates in coordination with the threaded slots 486. The oscillating surface 482 of the heel expansion platform 480 corresponds with an oscillating midfoot 414 on the bottom side of the base 410. The oscillating surface 482 of the heel expansion platform 480 and the oscillating midfoot 414 of the base 410 are configured to fit snugly together to provide additional securement of the heel expansion platform 480 to the base 410.

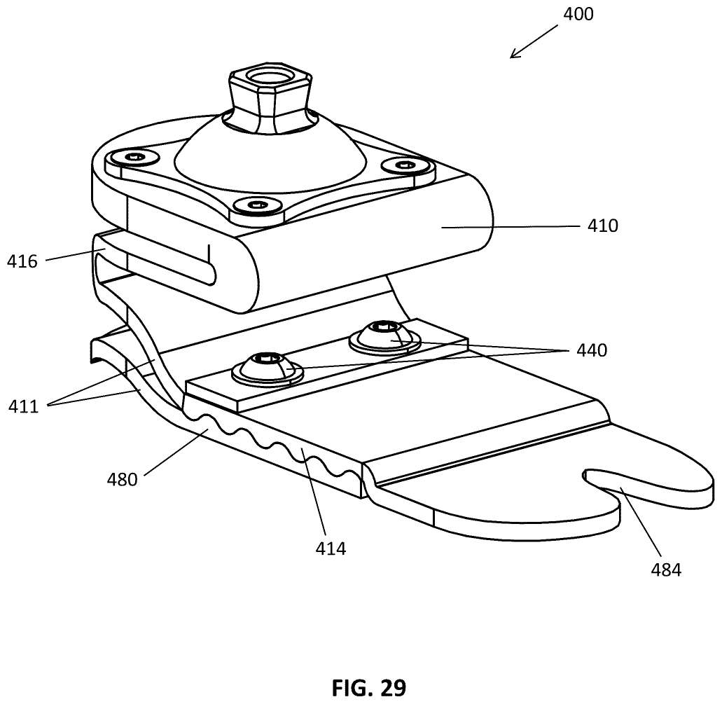

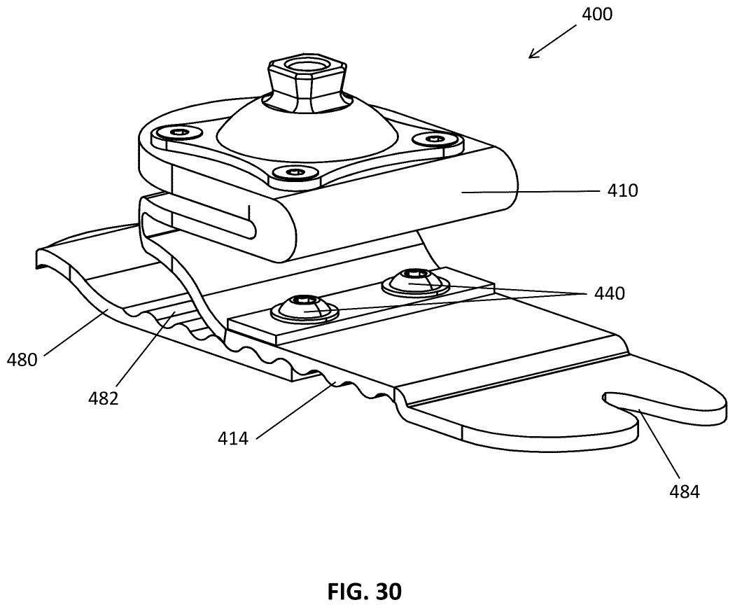

The slots 486 in the heel expansion platform 480 are incremented to correspond with a child's or user's shoe size to allow for proper fit in standard shoes as well as for adjustability so that the heel expansion platform 480 can be adjusted as a child or user grows. The fastener(s) 440 securing the heel expansion platform 480 to the base 410 can be loosened, allowing the heel expansion platform 480 to be adjusted to accommodate for change in size of the child's or user's foot, and tightened to keep the heel expansion platform 480 stationary. The fastener(s) 440 are threaded through at least one slot 444 of a fastener plate 442, through at least one slot 470 of the base 410, and into at least one threaded slot 486 of the heel expansion platform 480. FIG. 29 shows the heel expansion platform 480 of the foot 400 in a fully retracted or fully closed position. FIG. 30 shows the heel expansion platform 480 of the foot 400 in a fully expanded or fully opened position. In this example embodiment, the described design of the foot 400 allows for approximately an inch size length expansion; however, other expansion ranges may be used without departing from the scope of the disclosure. Further, any other mechanism for foot 400 size adjustment is hereby contemplated for use.

The foot 400 may be fabricated using methods of 3D printing with carbon fiber-reinforced filament or other suitable materials or methods. The components of the foot 400 may be fastened together with standard fasteners 440, such as metric screws and bolts.

The prosthesis 100 has many benefits and advantages including, but not limited to, being adjustable according to the growth of a user. The prosthesis 100 may be fit on a user-by-user basis by a certified prosthetist. Once properly fitted, the prosthesis 100 functions as an everyday walking aid, supporting activities such as walking, standing, climbing stairs, and other routine tasks.

The prosthesis 100 is all encompassing regarding adjustment in both height and width. Unlike traditional below-the-knee prosthetics which come in standard fixed sizes and designs, the disclosed prosthesis 100 offers a high degree of customization and adaptability. It can be tailored to fit the unique anatomy and requirements of individual users, allowing for a more comfortable and functional fit.

A key distinguishing feature of the disclosed prosthesis 100 is its ability to accommodate the growth of users, especially pediatric users, over time. Traditional prostheses may require frequent replacements as the child or user grows, resulting in significant costs, waste, and appointments. The disclosed prosthesis 100 minimizes the need for frequent replacements and allows for seamless adjustments as the user's body develops.

The prosthesis 100 components are designed to be adjustable in all relevant directions, while maintaining certain functional requirements. The prosthesis 100 facilitates typical biomechanics, including full range of motion at the knee/hip, and allows for proper gait without exertion. The design of the prosthesis 100 is also comparable in weight to existing solutions (less than 4 pounds). It is easy to use without professional clinical training and is usable without sophisticated tools. In addition, the prosthesis 100 is comfortable and durable. In terms of cost efficiency, the prosthesis 100 is affordable with materials that are simple to process and allow for the reduction of prosthesis 100 reiteration frequency.

These and other benefits and advantages of the prosthesis 100 are apparent from the specification and claims.

### Reference Numerals

100—Prosthesis

200—Socket

202—Lattice

204—Hardshell

206—Padding Layer

207—Pin (of Liner 208)

208—Liner

209—Lock (of Liner 208)

210—Replaceable top component, also called top component, top segment, proximal component, or proximal segment

215—Bottom component, also called bottom segment, distal component, or distal segment

216—Anterior windows

217—Posterior window

220—Dial system or dial

232—Inner Flaps (of Lattice 202)

234—Outer Flap (of Lattice 202)

236—Inner Flap (of Hardshell 204)

238—Outer Flap (of Hardshell 204)

240—Alignment Guides (of Lattice 202)

242—Alignment Guide Channels (of Lattice 202)

244—Opening (of Lattice 202)

246—Gap (of Lattice 202)

250—Base (of Lattice 202)

252—Shoulder (of Lattice 202)

254—Base (of Hardshell 204)

256—Shoulder (of Hardshell 204)

260—Front Recess (of Lattice 202)

262—Front Recess (of Hardshell 204)

264—Gap (of Hardshell 204)

266—Fastening Mechanism (of Hardshell 204)

300—Pylon

310—Pinion

320—Rack

322—Rack engaging surface

330—Guiderail

332—Bolt Head Recess (of Guiderail 330)

334—Nut Opening (of Guiderail 330)

340—Locking mechanism, also called lockrack

341—Notch

342—First rack engaging surface

343—Second rack engaging surface

344—Lockrack engaging surface

346—Grips (of lockrack 340)

348—Bolt Opening (of lockrack 340)

350—Attachment Member

360—Bolts

362—Nut (of Bolts 360)

400—Foot

410—Base

411—Split shank heel

412—Slotted tracks

414—Oscillating Midfoot (of Base 410)

16—Upper Portion (of Base 410)

420—Width-expansion layer or width-expansion platform

421—First width-expansion plate

422—Second width-expansion plate

423—Slot

430—Length-expansion layer or length-expansion platform

431—First length-expansion plate

432—Second length-expansion plate

440—Fastener

442—Fastener Plate

444—Slot (of Fastener Plate 442)

450—Medial expansion plate or medial expansion platform

460—Lateral expansion plate or lateral expansion platform

470—Slot (of Base 410)

480—Heel expansion platform

482—Oscillating surface (of Heel Expansion Platform 480)

484—Toe Notch

486—Slot (of Heel Expansion Platform 480)

488—First Connection Mechanism

490—Second Connection Mechanism

What is claimed:

1. An adjustable prosthesis, comprising:
a socket;
a pylon operably connected to the socket; and
a foot operably connected to the pylon.

2. The prosthesis of claim 1 wherein the socket comprises:
an adjustable fastening mechanism which causes the socket to be adjustable.

3. The prosthesis of claim 1 wherein the socket comprises:
a bottom component and a top component; and
wherein the top component is replaceable with a larger top component.

4. The prosthesis of claim 1 wherein the socket comprises:
a liner layer;
a padding layer;
a hardshell layer; and
a lattice layer.

5. The prosthesis of claim 1 wherein the pylon comprises:
a guiderail which causes the pylon to be adjustable.

6. The prosthesis of claim 1 wherein the pylon comprises:
a guiderail which causes the pylon to be adjustable;
wherein the guiderail comprises a rack and a lockrack; and
wherein the lockrack is configured to hold the rack in place when adjustment of the pylon is complete.

7. The prosthesis of claim 1 wherein the pylon comprises:
a guiderail which causes the pylon to be adjustable;
wherein the guiderail comprises a rack and a lockrack;
wherein the lockrack is configured to hold the rack in place when adjustment of the pylon is complete; and
wherein the pylon can be adjusted by a user by moving the rack.

8. The prosthesis of claim 1 wherein the pylon comprises:
a guiderail which causes the pylon to be adjustable;
wherein the guiderail comprises a rack and a lockrack;
wherein the rack comprises a surface with rack teeth;
wherein the lockrack comprises a surface with lockrack teeth; and
wherein the lockrack teeth are configured to engage the rack teeth thereby holding the rack in place.

9. The prosthesis of claim 1 wherein the pylon comprises:
a guiderail which causes the pylon to be adjustable;
wherein the guiderail comprises a rack and a lockrack;
wherein the rack comprises a surface with rack teeth;
wherein the lockrack comprises a surface with lockrack teeth;
wherein the lockrack teeth are configured to engage the rack teeth; and
wherein at least one fastener is configured to secure the lockrack to the guiderail and keep the lockrack teeth engaged with the rack teeth, thereby holding the rack in place.

10. The prosthesis of claim 1 wherein the foot comprises:
at least one expansion plate operably connected to a base of the foot; and
wherein the at least one expansion plate is configured to adapt to a user's foot size.

11. The prosthesis of claim 1 wherein the foot comprises:
a first expansion plate operably connected to a base of the foot and configured to extend to adapt to a user's foot; and
a second expansion plate operably connected to the base of the foot and configured to extend to adapt to the user's foot.

12. The prosthesis of claim 1 wherein the foot comprises:
at least one expansion plate; and
wherein the at least one expansion plate is operably connected to the foot via a plurality of slots on a base of the foot.

13. The prosthesis of claim 1 wherein the foot comprises:
at least one expansion plate;
wherein the at least one expansion plate is operably connected to the foot via a plurality of slots on a base of the foot; and
wherein at least one fastener is used to operably connect the at least one expansion plate to the foot.

14. A method of manufacturing an adjustable prosthesis, comprising the steps of:
operably attaching a socket to a pylon;
wherein the socket comprises a fastening mechanism which causes the socket to be adjustable;
wherein the pylon comprises a guiderail which causes the pylon to be adjustable;
operably attaching the pylon to a foot;
wherein the foot comprises;
at least one expansion plate operably connected to a base of the foot;
wherein the at least one expansion plate is configured to move and allow the foot to be adjustable to a user's foot size.

15. The method of claim 14 wherein operably attaching the socket to the pylon comprises the steps of;
attaching a liner layer to a residual limb of the user;
attaching a padding layer to the liner layer;
attaching a hardshell layer to the padding layer; and
attaching a lattice layer to the hardshell layer.

16. The method of claim 14 wherein the guiderail of the pylon further comprises;
a rack and a lockrack; and
wherein the lockrack is configured to secure the rack to the guiderail to allow the user to adjust the height of the pylon.

17. The method of claim 14 wherein the guiderail of the pylon further comprises:
a rack and a lockrack;
wherein the rack comprises rack teeth;
wherein the lockrack comprises lockrack teeth;
wherein the lockrack teeth are configured to engage the rack teeth, thereby holding the rack in place.

18. The method of claim 14 wherein the guiderail of the pylon further comprises:
a rack and a lockrack; and
wherein the lockrack is flush with the guiderail when the lockrack is secured to the rack.

19. The method of claim 14 wherein the foot further comprises:
a plurality of slots on the base of the foot; and
wherein at least one fastener is used to operably connect the at least one expansion plate to the foot.

20. An adjustable prosthesis, comprising:
an adjustable socket;
wherein the adjustable socket comprises an adjustable fastening mechanism such that the adjustable socket is adjustable to a user's specifications;
an adjustable pylon operably connected to the adjustable socket;
wherein the adjustable pylon comprises;
a guiderail comprising a rack and a lockrack which cause the adjustable pylon to be adjustable;
wherein the lockrack is configured to hold the rack in place when adjustment of the adjustable pylon is complete; and
wherein the lockrack attaches to the guiderail using at least one fastener capable of adjustment;
an adjustable foot operably connected to the adjustable pylon;
wherein the adjustable foot comprises;
a base;
a first expansion plate operably connected to the base and configured to extend lengthwise;
wherein the first expansion plate is configured to attach to the base at a plurality of slots; and
wherein the first expansion plate can be adjusted by the user to the user's specifications.
